(19) United States
(12) Patent Application Publication
(10) Pub. No.: US 2003/0028011 A1
(43) Pub. Date: Feb. 6, 2003

(54) Compositions and methods for determining susceptibility of hepatitis C virus to anti-viral drugs

(76) Inventors: Neil T. Parkin, Belmont, CA (US); Andrea Gamarnik, Chandler, AZ (US)

Correspondence Address: PENNIE AND EDMONDS, 1155 AVENUE OF THE AMERICAS, NEW YORK, NY, 100362711

(21) Appl. No.: 10/139,069

(22) Filed: May 3, 2002

Related U.S. Application Data
(63) Continuation-in-part of application No. 09/126,559, filed on Jul. 30, 1998.
(60) Provisional application No. 60/054,257, filed on Jul. 30, 1997.

(51) Int. Cl. C12Q 1/70; C12Q 1/68; C07H 21/04; C12N 15/00; C12N 15/09; C12N 15/63; C12N 15/70; C12N 15/74

(52) U.S. Cl. 536/23.72; 435/5; 435/6; 435/320.1

(57) ABSTRACT

The present invention provides methods for determining the susceptibility of a pathogenic flavivirus to anti-viral compounds. This invention also provides methods for determining anti-viral drug susceptibility in a patient infected with a flavivirus. This invention also provides a method for evaluating the biological effectiveness of a candidate anti-viral drug compound. The methods are useful for identifying effective drug regimens for the treatment of flaviviral infections, and identifying and assessing the biological effectiveness of potential therapeutic compounds. Compositions including resistance test vectors and host cells transformed with the resistance test vectors are provided.

This application is a continuation-in-part of U.S. Ser. No. 09/126,559, filed Jul. 30, 1998, which claim the benefit under 35 U.S.C. §119(e) of U.S. Provisional Application No. 60/054,257, filed Jul. 30, 1997. The above applications are incorporated herein by reference in their entireties.

### 1. FIELD OF INVENTION

This invention relates to methods and compositions for determining the susceptibility of a pathogenic virus to anti-viral compounds. The methods are useful for identifying effective drug regimens for the treatment of viral infections, and identifying and assessing the biological effectiveness of potential therapeutic compounds.

### 2. BACKGROUND OF THE INVENTION

Infection with hepatitis C virus (“HCV”) is an important cause of chronic liver disease in North America and the world and, prior to its identification, represented the major cause of transfusion-associated hepatitis. Current estimates of the number of infected individuals range from 3 to 4 million in the United States (Alter et al., 1999, N Engl J Med 341:556-62), and nearly 170 million worldwide (World Health Organization. 1997, Hepatitis C: Global Prevalence. Weekly Epidemiological Record 72:341). This represents an approximately fourfold higher prevalence rate compared to human immunodeficiency virus type 1 (HIV-1) infection (Karon et al., 1996, JAMA 276:126-31). Drug resistance in pathogenic viruses is a substantial problem. For example, approximately 140 HIV drug resistance mutations to various anti-viral agents have been identified to date (Mellors et al., 1995, Intnl. Antiviral News, supplement and Condra et al., 1996, J Virol. 70:8270-8276), and approximately 20 human cytomegalovirus (HCMV) drug resistance mutations to various anti-viral agents have been identified to date (Erice, 1999, Clin Microbiol Rev 12(2):286-297; Chou, 1999, Transpl Infect Dis. 1(2):105-14; Arens, 2001, J Clin Virol. 22(1):11-29; Emery, 2001, J Clin Virol. 21 (3):223-228).

The emergence of a population of drug-resistant viruses results from the survival and selective proliferation of a previously existing sub-population that randomly emerges in the absence of selective pressure. For example, with approximately 10¹⁰ new virions being generated daily during HIV infection (Ho et al., 1995, Nature 373:123-126), a mutation rate of 10⁻⁴ to 10⁻⁵ per nucleotide guarantees the preexistence of almost any single point mutation at any time point during HIV infection.

The problem of drug resistance may be particularly acute for HCV. Based on comparison with other RNA derived RRNA Polymerases (“RDRPs”) (Steinhauer et al., 1987, Annu Rev Microbiol 41:409-33) and the quasispecies diversity observed in HCV infected patients (Cabot et al., 2001, J Virol 75:12005-12013; Martell et al., 1992, J Virol 66:3225-3229), it is generally accepted that the HCV RDRP has relatively poor fidelity. Coupled with a high replication rate (10¹² viral particles produced per day), the potential for the generation of drug resistant variants is high (Neumann et al., 1998, Science 282:103-107). When anti-viral therapy is incompletely suppressive, new variants with additional mutations can be generated and mutants with increased resistance may emerge. By analogy to the clinical situation with HIV-1, it is anticipated that HCV will develop resistance to any given drug with equal or greater case. There are two types of assays for assessing the susceptibility of a patient infected with a virus to an anti-viral drug: genotypic assays and phenotypic assays. Genotypic assays determine the nucleotide sequence of the predominant population of viruses present at the time of sampling and predict drug susceptibility based on clinical or laboratory data correlating the presence of certain mutations to treatment response or in vitro susceptibility (Deeks et al., 1997, Lancet 349:1489-1490). In contrast, phenotypic assays provide a direct, quantitative measure of drug susceptibility and are able to identify the drugs that are most likely to be effective for treating the patient.

There are no well established drug susceptibility assays for HCV currently available. However, several investigators have devised chimeric virus systems containing the HCV NS3 protease such that replication of the chimera, or expression of a reporter gene, is dependent on NS3 activity. See, e.g., Hirowatari et al, 1995, Anal. Biochem., 225:113; Hahm et al., 1996, Virology 226:318; Filocamo et al., 1997, J. Virol. 71:1417; Lai et al., 2000, J. Virol. 74:6339. These systems were devised with the limited focus of studying various aspects of NS3 function and its cofactor, NS4A, and to serve as prototypes for cell-based drug screening assays.

The pathogenicity of HCV coupled with the lack of well established drug susceptibility assays for HCV warrants an urgent need in the art for a phenotypic drug susceptibility assay for HCV.

### 3. SUMMARY OF THE INVENTION

The present invention provides methods and compositions for determining whether a flavivirus is affected by a compound. The flavivirus may be derived from a patient making it useful in a wide range of laboratory or clinical settings.

In one aspect, the present invention provides a method for determining whether a test hepatitis C virus (HCV) has an altered susceptibility to a compound, comprising a) contacting a test host cell with the compound, wherein the test host cell comprises a test HCV-derived nucleic acid and an indicator gene, the activity of the indicator gene is affected by the activity of the test HCV-derived nucleic acid such that a change in the activity of the test HCV-derived nucleic acid results in a change in the activity of the indicator gene, and the compound directly or indirectly targets the test HCV-derived nucleic acid or a protein it encodes, and b) detecting the activity of the indicator gene,wherein a difference in the activity of the indicator gene in the test host cell contacted with the compound relative to the activity of the indicator gene in a reference host cell contacted with the compound and comprising the indicator gene and a reference HCV-derived nucleic acid, the reference HCV-derived nucleic acid being similar to the test HCV-derived nucleic acid but differing therefrom at one or more nucleotides, indicates that the test HCV has an altered susceptibility to the compound.

In another aspect, the present invention provides a method for determining whether a hepatitis C virus (HCV) is susceptible to an anti-hepatitis C compound, comprising a) contacting a host cell with the compound, wherein the host cell comprises both a nucleic acid derived from the virus and an indicator gene, the activity of the indicator gene is affected by the activity of the HCV-derived nucleic acid such that a change in the activity of the HCV-derived nucleic acid results in a change in the activity of the indicator gene, and the compound directly or indirectly targets the HCV-derived nucleic acid or a protein it encodes, and b) detecting the activity of the indicator gene, wherein a change in the activity of the indicator gene in the host cell contacted with the compound relative to the activity of the indicator gene in the host cell not contacted with the compound indicates that the virus is susceptible to the anti-hepatitis C compound.

In one embodiment, the compound is an anti-viral drug. In another embodiment, the anti-viral drug is a drug that targets one, two or more viral proteins encoded by the HCV-derived nucleic acid, including, but not limited to, C, E1, E2, NS2, NS3, NS4A, NS4B, NS5A or NS5B. In another embodiment, the compound is an anti-hepatitis C compound or a biomolecule. In another embodiment, the biomolecule is a protein, nucleic acid, RNA or DNA, for example. In another embodiment, the compound increases the activity of the indicator gene. In yet another embodiment, the compound decreases the activity of the indicator gene.

In another embodiment, the HCV-derived nucleic acid is a functional viral sequence and comprise an IRES, or encodes a protein, such as C, E1, E2, NS2, NS3, NS4A, NS4B, NS5A or NS5B. In another embodiment, the HCV-derived nucleic acid is present in a viral vector. In another embodiment, the viral vector is a genomic, subgenomic, indicator or resistance test viral vector. In another embodiment, the viral vector comprises genes encoding one or more of C, E1, E2, NS2, NS3, NS4A, NS4B, NS5A or NS5B. In another embodiment, the HCV-derived nucleic acid is RNA or DNA.

In another embodiment, the method is a single cell assay. In another embodiment, the method is a two cell assay.

In another embodiment, the change in the activity of the indicator gene is a reduction.

In another embodiment, the change in the activity of the indicator gene is an increase. In another embodiment, the altered susceptibility is a decreased susceptibility. In another embodiment, the altered susceptibility is a increased susceptibility. In another embodiment, the increased activity of the test host cell relative to the reference host cell indicates the HCV-derived nucleic acid has decreased susceptibility.

In another embodiment, the HCV-derived nucleic acid is a patient-derived viral segment.

In another aspect, the invention provides a method for determining whether a patient infected with hepatitis C virus is likely to be susceptible to treatment with an anti-hepatitis C compound comprising a) contacting a test host cell with the compound, wherein the test host cell comprises a patient-derived viral segment and an indicator gene, the activity of the indicator gene is affected by the activity of the patient-derived viral segment such that a change in the activity of the patient-derived viral segment results in a change in the activity of the indicator gene, and the compound directly or indirectly targets the patient-derived viral segment or a protein it encodes, and b) detecting the activity of the indicator gene, wherein an increase in the activity of the indicator gene in the test host cell contacted with the compound relative to the activity of the indicator gene in a reference host cell contacted with the compound and comprising the indicator gene and a reference viral segment, the reference viral segment being similar to the patient-derived viral segment but differing therefrom at one or more nucleotides, indicates that the patient is less likely to be susceptible to treatment with the compound.

In one embodiment, the compound is an anti-viral drug. In another embodiment, the anti-viral drug is a drug that targets one, two or more viral proteins encoded by the HCV-derived nucleic acid, including, but not limited to, C, E1, E2, NS2, NS3, NS4A, NS4B, NS5A or NS5B. In another embodiment, the compound is an anti-hepatitis C compound or a biomolecule. In another embodiment, the biomolecule is a protein, nucleic acid, RNA or DNA, for example. In another embodiment, the compound increases the activity of the indicator gene. In yet another embodiment, the compound decreases the activity of the indicator gene.

In another embodiment, the indicator gene and the HCV-derived nucleic acid are part of the same nucleic acid and are oriented in the same sense. In another embodiment, the indicator gene and HCV-derived nucleic acid are part of the same nucleic acid and are oriented in the opposite sense. In another embodiment, the indicator gene and the patient-derived viral segment are part of the same nucleic acid and are oriented in the same sense. In yet another embodiment, the indicator gene and the patient-derived viral segment are part of the same nucleic acid and are oriented in the opposite sense.

In another embodiment, the patient-derived viral segment encodes one, two or more proteins that are the target of one or more anti-viral drugs. In another embodiment, the patient-derived viral segment is a functional viral sequence. In another embodiment, the functional viral sequence is IRES. In another embodiment, the patient-derived viral segment encodes a protein or peptide. In a further embodiment, the encoded protein is a protein involved in the replication of viral RNA or involved in the processing of the viral polyprotein. In another embodiment, the patient-derived viral segment encodes C, E 1, E2, NS2, NS3, NS4A, NS4B, NS5A or NS5B. In another embodiment, the patient-derived viral segment is present in a viral vector, as part of a genomic viral vector, as part of a subgenomic viral vector or as part of an indicator gene viral vector.

In another embodiment the vector comprises genes encoding one or more of C, E1, E2, NS2, NS3, NS4A, NS4B, NS5A or NS5B. In another embodiment, the vector is comprised of RNA or DNA. In yet another embodiment, the vector is present as part of a resistence test vector.

In another embodiment, the host cell is a packaging host cell, a resistance test vector host cell or a target host cell. In another embodiment, the host cell is a mammalian cell, a human cell, a liver cell, a hepatoma cell or hepatocyte, for example, HepG2, Huh7, Hep3B, PLC/PRF/5, Huh6, HLE, SK-Hepl, HepT1, HepT3 and HLF.

In another embodiment, the indicator gene is a functional or non-functional indicator gene. In another embodiment, the activity of the indicator gene provides color, light, radioactive emissions, fluorescence or allow the host cell to grow in a medium lacking a nutrient or containing a toxin. In another embodiment, the activity of the indicator gene is detected by radioimmunoassay or fluorescent activated cell sorting. In another embodiment, the indicator gene is a luciferase gene, β-lactamase gene or an E. coli lacZ gene, alkaline phosphatase, a fluorescent protein, including red and green fluorescent protein, chloramphenicol acetyltransferase, a secreted protein or a cell surface protein. In another embodiment, the indicator gene is integrated into the host cell genome, including, but not limited to, integration into the host cell genome as part of a vector. In another embodiment, the indicator gene is comprised of RNA or DNA.

In another embodiment, the indicator gene is present with either the HCV-derived nucleic acid or the patient-derived viral segment as part of a resistance test vector.

In another embodiment, the method uses a viral segment from a patient who is undergoing or has undergone a prior treatment, for example, an anti-hepatitis C treatment.

In another aspect, the invention provides a vector. In one embodiment, the vector comprises a patient-derived viral segment and an indicator gene, wherein the patient-derived viral segment comprises all or part of a hepatitis C virus genome. In another embodiment, the patient-derived viral segment and the indicator gene are selected from those described above. The vector may also be, for example, vectors according to FIGS. 3a, 3b, 3c, 3d, 4, 5, 6, 7, 9, 10, 12 and 14 wherein the vector comprises a patient-derived viral segment.

In another aspect, the invention includes a cell comprising a vector. The cell may be, for example, a mammalian cell, a human cell, a liver cell, a hepatoma or a hepatocyte, for example, HepG2, Huh7, Hep3B, PLC/PRF/5, Huh6, HLE, SK-Hep1, HepT1, HepT3 and HLF. The vector can be, for example, one of the vectors described above comprising a patient-derived viral segment.

### 4. BRIEF DESCRIPTION OF THE FIGURES

FIG. 1 is a schematic drawing of the approximately 9.5 kb HCV RNA.

FIG. 2 is a schematic drawing of the replication cycle of HCV.

FIG. 3A is a diagrammatic representation of the resistance test vector (pXHCV-luc, where X is either CMV or T7).

FIG. 3B is a diagrammatic representation of a resistance test vector (pCMVHCV-luc) comprising the CMV IE promoter and SV40 polyadenylation signal.

FIG. 3C is a diagrammatic representation of a resistance test vector (pT7HCV-luc1) comprising the T7 RNA polymerase promoter and T7 RNA polymerase terminator.

FIG. 3D is a diagrammatic representation of a resistance test vector (pT7HCV-luc2) comprising the T7 RNA polymerase promoter and a restriction site placed at the 3′ end for linearization of the DNA prior to transcription in vitro.

FIG. 4 is a diagrammatic representation of a resistance test vector (pXHCV-IRESluc) comprising an IRES element for luciferase translation.

FIG. 5 is a diagrammatic representation of a resistance test vectors (PXHCV and pXIRESluc) comprising a positive sense luciferase RNA minigenome.

FIG. 6 is a diagrammatic representation of a resistance test vector (pXHCV-ASIRESluc) comprising an antisense RNA minigenome.

FIG. 7 is a diagrammatic representation of a resistance test vectors (pXluc-NSHCV and pXSHCV) expressing defective genomic RNAs.

FIG. 8 is a diagrammatic representation of the BVDV genome.

FIG. 9 is a diagrammatic representation of a resistance test vector pXBVDV(HCVNS5B) luc.

FIG. 10 is a diagrammatic representation of a neomycin resistance-conferring replicon.

FIG. 11 shows the establishment of neomycin (G418)-resistant colonies in different replicons.

FIG. 12 is a diagrammatic representation of a replicon comprising the luciferase indicator gene.

FIG. 13 shows Luciferase activity after electroporation of luc replicons in Huh-7 cells.

FIG. 14 is a diagrammatic representation of a non-functional indicator gene construction.

### 5. DETAILED DESCRIPTION OF THE INVENTION

The present invention provides methods for assaying the susceptibility of a Flaviviridae virus to an anti-viral treatment, methods for assaying the effectiveness of an anti-viral treatment of an individual infected with a Flaviviridae virus, and methods of monitoring the clinical progression of Flaviviridae viral infection in individuals receiving anti-viral treatment. In another aspect, the present invention also provides compositions including resistance test vectors, indicator genes, and host cells transformed with the resistance test vectors.

5.1 Abbreviations and Definitions

“HCV” is an abbreviation for “hepatitis C virus.”

“HIV” is an abbreviation for “human immunodeficiency virus.”

“EMCV” is an abbreviation for “encephalomyocarditis virus.”

“IRES” is an abbreviation for “internal ribosome entry site.”

“UTR” is an abbreviation for “untranslated region.” A 3′ UTR occurs at the 3′ end and a 5′ UTR occurs at the 5′ end of a viral genome.

“RDRP” is an abbreviation for “RNA-dependent RNA polymerase.”

“IG” is an abbreviation for “indicator gene.”

“IGVV” is an abbreviation for “indicator gene viral vector.”

“GFP” is an abbreviation for “green fluorescent protein.”

“ORF” is an abbreviation for “open reading frame.”

“BVDV” is an abbreviation for “bovine viral diarrheal virus.”

“PSAS” is an abbreviation for “patient sequence acceptor site.”

“PCR” is an abbreviation for “polymerase chain reaction.”

“PDS” is an abbreviation for “patient derived segment.”

The amino acid notations used herein for the twenty genetically encoded L-amino acids are conventional and are as follows:

| | One-Letter | Three Letter |
|---|---|---|
| Amino Acid | Abbreviation | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Gln |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Unless noted otherwise, when polypeptide sequences are presented as a series of one-letter and/or three-letter abbreviations, the sequences are presented in the N->C direction, in accordance with common practice. The amino acid positions are numbered based on the full-length sequence of the protein from which the region encompassing the mutation is derived. Representations of nucleotides and point mutations in DNA sequences are analogous.

The abbreviations used throughout the specification to refer to nucleic acids comprising specific nucleobase sequences are the conventional one-letter abbreviations.

Thus, when included in a nucleic acid, the naturally occurring encoding nucleobases are abbreviated as follows: adenine (A), guanine (G), cytosine (C), thymine (T) and uracil (U). Also, unless specified otherwise, nucleic acid sequences that are represented as a series of one-letter abbreviations are presented in the 5′->3′ direction.

Two or more nucleic acid or amino acid sequences are “similar” if they share significant sequence similarity. For example, the HCV NS5A gene from two different HCV isolates are “similar” even though they might be from different types, sub-types, genetic variants or mutant strains of HCV, and even though their nucleotide sequences might differ from each by one or more insertions, deletions or missense mutations.

“ICₙ” refers to Inhibitory Concentration. It is the concentration of drug in the patient's blood or in vitro needed to suppress the reproduction of a disease-causing microorganism (such as HCV) by n %. Thus, “IC₅₀” refers to the concentration of an anti-viral agent at which virus replication is inhibited by 50% of the level observed in the absence of the drug. “Patient IC₅₀” refers to the drug concentration required to inhibit replication of the virus from a patient by 50% and “reference IC₅₀” refers to the drug concentration required to inhibit replication of a reference or wild-type virus by 50%. Similarly, “IC₉₀” refers to the concentration of an anti-viral agent at which 90% of virus replication is inhibited.

“Susceptibility” refers to a virus' response to a particular drug. A virus that has decreased or reduced susceptibility to a drug has an increased resistance or decreased sensitivity to the drug as compared to a reference or wild-type virus. A virus that has increased or enhanced or greater susceptibility to a drug has an increased sensitivity or decreased resistance to the drug.

A “phenotypic assay” is a test that measures the sensitivity of a virus (such as HCV) to a specific anti-viral agent.

A “mutation” is a change in an amino acid sequence or in a corresponding nucleic acid sequence relative to a reference protein or nucleic acid. Although the amino acid sequence of a peptide can be determined directly by, for example, Edman degradation or mass spectroscopy, more typically, the amino sequence of a peptide is inferred from the nucleotide sequence of a nucleic acid that encodes the peptide. Any method for determining the sequence of a nucleic acid known in the art can be used, for example, Maxam-Gilbert sequencing (Maxam et al., 1980, Methods in Enzymology 65:499), dideoxy sequencing (Sanger et al., 1977, Proc. Natl. Acad. Sci. USA 74:5463) or hybridization-based approaches (see e.g., Sambrook et al., 1989, Molecular Cloning: A Laboratory Manual, Cold Spring Harbor Laboratory, NY and Ausubel et al., 2002, Current Protocols in Molecular Biology, Greene Publishing Associates and Wiley Interscience, N.Y.).

A “mutant” is a virus, gene or protein having a sequence that has one or more changes relative to a reference virus, gene or protein.

The terms “peptide” “polypeptide” and “protein” are used interchangeably throughout.

The terms “nucleotide,” “oligonucleotide” and “nucleic acid” are used interchangeably throughout.

The terms “drug,” “anti-viral,” “anti-retroviral,” “anti-viral agent,” “anti-retroviral agent,” “anti-viral compound,” and “anti-retroviral compound” are used interchangeably throughout and are meant to encompass all chemical compounds as well as biomolecules, examples of which include, but are not limited to, peptides and nucleic acid molecules.

“Host Cells” are cells comprising all or part of a resistance test vector or other nucleic acid of the invention.

“Resistance test vector (“RTV”)” refers to one or more nucleic acids which taken together comprise a patient-derived segment and an indicator gene. One or more nucleic acids comprising the vector can be integrated into the host cell's genome (e.g., the patient-derived segment can be present in an extrachromosomal nucleic acid and the indicator gene integrated into a host cell's genome). An RTV comprising more than one nucleic acid is referred to herein as a “resistance test vector system” for purposes of clarity but is nevertheless understood to be a resistance test vector. “Patient-derived segment” refers to one or more viral segments obtained from a patient. The term encompasses segments derived from human and various animal species. Patient-derived segments can also be incorporated into resistance test vectors.

“Patient sequence acceptor site” refers to a site in a vector or other nucleic acid for insertion of patient-derived segments, e.g., a restriction site, a polylinker, or a homologous recombination site.

“Viral segment” refers to any viral sequence, or viral gene encoding a gene product (e.g., a protein), that is the target of an anti-viral drug.

“Functional viral sequence” refers to any nucleic acid sequence (DNA or RNA) with functional activity such as enhancers, promoters, polyadenylation sites, sites of action of trans-acting factors, internal ribosome entry sites (IRES), translation frameshift sites, packaging sequences, integration sequences, splicing sequences etc.

A “packaging expression vector” provides the factors, such as packaging proteins (e.g., structural proteins such as core and envelope polypeptides), enzymes required for replication (e.g., polymerases and proteases), trans-acting factors, genes, etc., required by a corresponding packaging or replication-defective virus to replicate and form particles.

“Indicator or indicator gene” refers to a nucleic acid encoding a protein, DNA or RNA structure that either directly or through a reaction gives rise to a measurable or noticeable aspect, e.g., a color or light of a detectable wavelength , or, in the case of DNA or RNA used as an indicator, a change or generation of a specific DNA or RNA structure. The term “indicator gene” also includes, e.g., a selection gene, also referred to as a selectable marker, for certain embodiments of the invention. Examples of selectable markers include nucleic acids whose presence in a cell allows the cell to survive or grow under conditions where a cell without the selectable marker will die or not grow, e.g., in medium lacking a nutrient or containing a toxin. The indicator gene may be either functional or non-functional, but in each case the expression of the indicator gene in the target cell is ultimately dependent upon the action of the patient-derived segment.

“Indicator gene activity” refers to the property of the indicator gene or the RNA or protein it encodes that produces the measureable or noticeable aspect.

“Indicator gene viral vector” refers to one or more vectors comprising an indicator gene and its control elements and one or more viral genes.

An “indicator gene cassette” comprises an indicator gene and, optionally, one or more control elements.

“Viral vector” refers to a vector comprising any or all of the following: viral genes encoding a gene product, control sequences, viral packaging sequences. The viral vector may additionally include one or more viral segments one or more of which may be the target of an anti-viral drug.

A “genomic viral vector” refers to a vector which can comprise a deletion of one or more viral genes to render the virus replication incompetent, but which otherwise preserves the mRNA expression and processing characteristics of the complete virus.

A “subgenomic viral vector” refers to a vector comprising the coding region of one or more viral genes which can encode the protein(s) that are the target(s) of the anti-viral drug.

The following flow chart illustrates non-limiting examples of the various vectors and host cells that may be used in this invention.

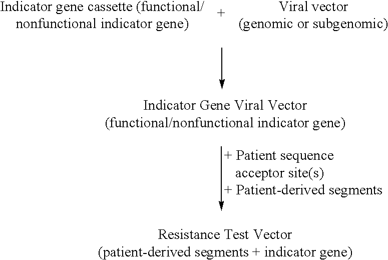

5.2 HCV Biology

HCV is currently the only known member of a distinct genus called hepacivirus in the family Flaviviridae, which includes the flaviviruses, the animal pathogenic pestiviruses and, it is thought, the recently cloned GB virus A (GBV-A), GBV-B and GBV-C/hepatitis G viruses (Murphy et al., 1995, Virus Taxonomy. Sixth Report of the International Committee on Taxonomy of Viruses, pp. 424-26, Vienna & New York: Springer-Verlag). It is an enveloped virus with a positive stranded, non-segmented RNA genome of about 9600 nucleotides in length (reviewed in Bartenschlager et al., 2000, J Gen Virol 81:1631-48). Multiple types and subtypes of HCV have been described based on serological studies and phylogenetic analysis of nucleotide sequence data, including, e.g., 1a, 1b, 1c, 2a, 2b, 2c, 3a, 3b, 4a, 5a and 6a (using the classification system of Simmonds et al., 1994, Hepatology 19:1321-24; reviewed in Zein, 2000, Clin. Microbiol. Rev. 13:223-235). Relative differences in type and subtype prevalence based on the geographical region have been observed (Zein, 2000, Clin. Microbiol. Rev. 13:223-235).

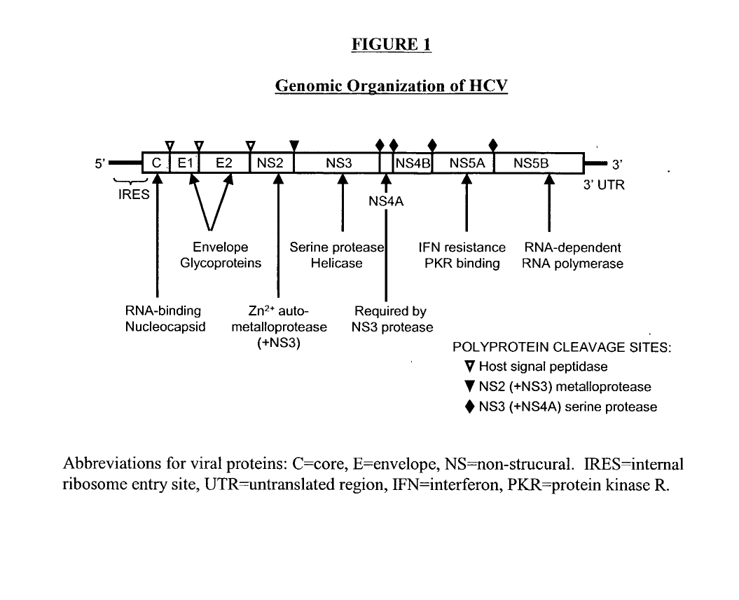

FIG. 1 is a schematic diagram of the approximately 9.5 kb HCV RNA. The order of the individual HCV proteins is indicated in the HCV polyprotein, with putative functions associated indicated below. Cleavage sites for proteolytic processing are indicated by open triangles for host signal peptidase, black triangles for NS2/3, and black diamonds for NS3/4A. The internal ribosome entry site (IRES) is located at the 5′ end of the RNA and comprises the entire untranslated region (UTR) and some sequences at the beginning of the C ORF. The 3′ end of the RNA comprises either a poly(A) or poly(U) tail, depending on the type of HCV.

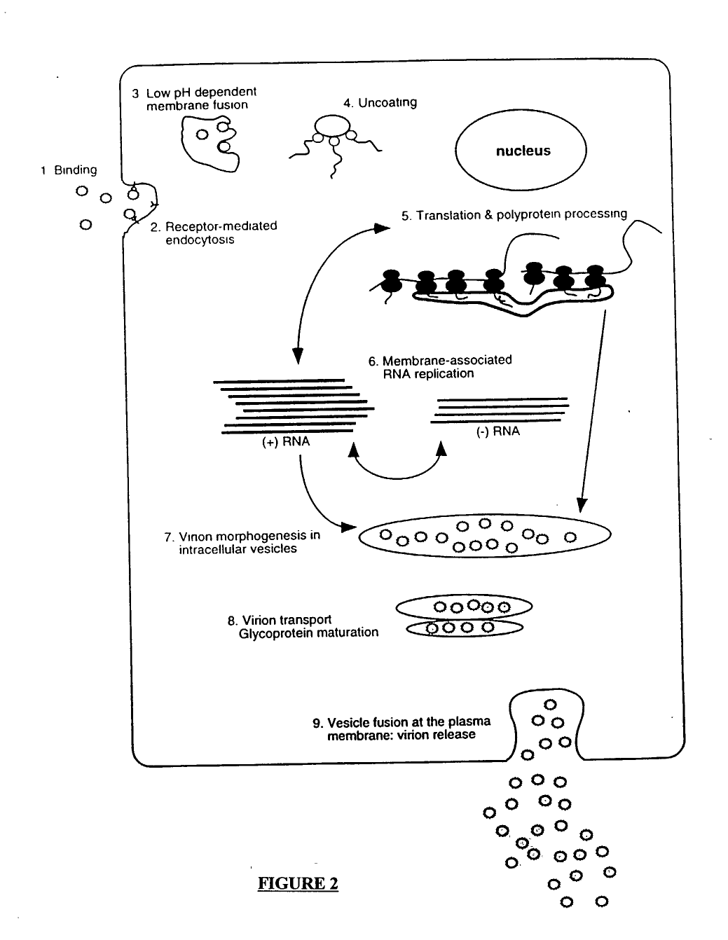

As with other flaviviruses, HCV replication is characterized by the production of a large (approximately 3000 amino acid) polyprotein that is subsequently processed by a variety of cellular and viral proteases to generate the structural (core (C), envelope (E1 and E2)) and non-structural (NS2, NS3, NS4, NS5) proteins. Replication of the RNA genome takes place in association with membranes in the cytoplasm of infected cells and proceeds via a minus strand RNA intermediate catalyzed by NS5B (FIG. 2). FIG. 2 is a schematic drawing of the replication cycle of HCV. Virions bind to the cell surface, via a specific interaction between a viral surface glycoprotein and a cell surface receptor (“1” in FIG. 2). Following receptor-mediated endocytosis (“2” in FIG. 2) and low pH dependent membrane fusion (“3” in FIG. 2), the nucleocapsid core is released into the cytoplasm (“4” in FIG. 2). Virion RNA is translated in close association with the endoplasmic reticulum (“ER”), and the polyprotein is processed by specific endoproteolytic cleavages mediated by host signal peptidase in the ER, or one of two viral proteases (“5” in FIG. 2). After enough of the non-structural proteins have been produced, the viral RNA is replicated through a antisense strand intermediate, to generate more positive sense RNA for translation and packaging into new virions (“6” in FIG. 2). Structural proteins and RNA assemble to form new viral particles which bud into the ER (“7” in FIG. 2) and are secreted via the cellular pathway (“8” and “9” in FIG. 2) to release the progeny virions.

5.3 Molecules of the Invention

5.3.1 Resistance Test Vectors

In one aspect, the invention provides a resistance test vector (“RTV”). An RTV refers to one or more nucleic acids which taken together comprise a patient-derived segment and an indicator gene. In one embodiment, the resistance test vector is made by insertion of a patient-derived segment into an indicator gene viral vector. In another embodiment, the resistance test vector is made by insertion of a patient-derived segment into a packaging vector while the indicator gene is present in a second vector, for example, an indicator gene viral vector (see below). In another embodiment, one or more nucleic acid molecules of the RTV are are integrated into a host cell's genome.

Resistance test vectors can be prepared by modifying an indicator gene viral vector, or packaging vector, by introducing a patient sequence acceptor site, amplifying or cloning patient-derived segments and inserting the amplified or cloned sequences precisely into indicator gene viral vectors, or packaging vectors, at the patient sequence acceptor sites. Resistance test vectors that are constructed from indicator gene viral vectors can be derived from genomic viral vectors or subgenomic viral vectors and an indicator gene cassette, each of which is described below. Resistance test vector systems that are constructed from packaging indicator vectors can be derived from genomic packaging vectors or subgenomic packaging vectors and an indicator gene cassette, each of which is described below. In addition, the indicator gene may be present in a host cell as, for example, an extrachromosomal element or integrated into the host cell's genome.

Resistance test vectors can then be introduced into a host cell. Alternatively, a resistance test vector can be prepared, for example, by introducing a patient sequence acceptor site into a packaging vector, amplifying or cloning patient-derived segments and inserting the amplified or cloned sequences precisely into the packaging vector at the patient sequence acceptor site and co-transfecting this packaging vector with an indicator gene viral vector.

In one embodiment, the resistance test vector is introduced into packaging host cells together with packaging expression vectors, as defined below, to produce resistance test vector viral particles that are used in drug susceptibility tests that are referred to herein as a “particle-based test.” In an alternative embodiment, the resistance test vector is introduced into a host cell in the absence of packaging expression vectors to carry out a drug susceptibility test that is referred to herein as a “non-particle-based test.”

A packaging expression vector provides the factors, such as packaging proteins (e.g., structural proteins such as core and envelope polypeptides), trans-acting factors, genes, etc., required by a replication-defective virus. In such a situation, a replication-competent viral genome is enfeebled so that it cannot replicate on its own. This means that, although the packaging expression vector can produce the trans-acting or missing genes required to rescue a defective viral genome present in a cell comprising the enfeebled genome, the enfeebled genome cannot rescue itself.

Resistance test vectors can be expressed using any method known in the art. For eaxmple, since the genome length RNA of flaviviruses is infectious, HCV vectors can be in the form of a cDNA construct containing a promoter for the T7 RNA polymerase at the 5′ end, and a T7 polymerase terminator sequence at the 3′ end or a control element specifying precise 3′ end formation, such as the hepatitis delta virus ribozyme (Perotta et al., 1991, Nature 350:434-36; Chowrira et al., 1994, J. Biol. Chem. 269: 25864; Wadkins et al., 2002, Cell Mol. Life Sci. 59:112-25). Thus RNA can be synthesized in large quantities in vitro and transfected into cells. An alternative approach is to transfect DNA constructs, which contain a strong eucaryotic promoter (such as the CMV IE promoter), directly into cells. A third transfection strategy is DNA transfection of constructs containing a T7 RNA polymerase promoter at the 5′ end, and a T7 RNA polymerase terminator at the 3′ end or a control element specifying precise 3′ end formation, such as the hepatitis delta virus ribozyme, into cells which express the T7 RNA polymerase. Expression of the polymerase may be achieved by various means, perhaps the most efficient of these being the infection of the transfected cells with a recombinant T7 polymerase/vaccinia virus (Fuerst et al., 1986, PNAS 83, 8122-8126).

5.3.2 Patient-Derived Segment and Patient Sequence Acceptor Site

A patient-derived segment (“PDS”) is one or more viral segments obtained from a patient. The PDS can be derived using any means known in the art, including, but not limited to, molecular cloning or amplification (e.g., by polymerase chain reaction) of a population of patient-derived segments using viral DNA or complementary DNA (“cDNA”) prepared from viral RNA present in the cells (e.g., peripheral blood mononuclear cells), serum or other bodily fluids of infected patients. In one embodiment, the PDS is obtained directly from a patient. When a viral segment is “obtained directly” from a patient it is obtained without passage of the virus through culture, or if the virus is cultured, then by a minimum number of passages or otherwise reducing the selection of mutations in culture. The term patient refers both to human and various animal species. Such species include, but are not limited to chimpanzees and other primates, horses, cattle, cats and dogs. Patient-derived segments also can be incorporated into resistance test vectors using any of several alternative, e.g., cloning techniques. Examples of cloning techniques that can be used include, but are not limited to, cloning via the introduction of class II restriction sites into the plasmid backbone and/or the patient-derived segments, uracil DNA glycosylase primer cloning, site specific recombination, or exonuclease overhang cloning.

The patient-derived segment can be obtained by any method of molecular cloning or gene amplification, or modifications thereof (see e.g., Sambrook et al., 1989, Molecular Cloning: A Laboratory Manual, Cold Spring Harbor Laboratory, NY and Ausubel et al., 2002, Current Protocols in Molecular Biology, Greene Publishing Associates and Wiley Interscience, N.Y.), by introducing one or more patient sequence acceptor sites, as described below, at the ends of the patient-derived segment to be introduced into the resistance test vector. For example, in a gene amplification method such as PCR, restriction sites corresponding to the patient-sequence acceptor sites can be incorporated at the ends of the primers used in the PCR reaction. Similarly, in a molecular cloning method such as cDNA cloning, said restriction sites can be incorporated at the ends of the primers used for first or second strand cDNA synthesis, or in a method such as primer-repair of DNA, whether cloned or uncloned DNA, said restriction sites can be incorporated into the primers used for the repair reaction. The patient sequence acceptor sites and primers are designed to improve the representation of patient-derived segments. Sets of resistance test vectors having one or more designed patient sequence acceptor sites provide representation of patient-derived segments that would be under-represented in one resistance test vector alone.

A patient sequence acceptor site is a site in a vector for insertion of a patient-derived segment. These sites can be of various types, including, but not limited to, unique restriction sites introduced by site-directed mutagenesis into a vector, naturally occurring unique restriction sites in the vector, selected sites into which a patient-derived segment may be inserted using alternative cloning methods (e.g., UDG cloning, exonuclease overhang cloning), or site specific recombination target sites. In one embodiment the patient sequence acceptor site is introduced into the indicator gene viral vector. The patient sequence acceptor site is preferably located within or near the coding region of the viral protein which is the target of the anti-viral drug. The viral sequences used for the introduction of a patient sequence acceptor site is preferably chosen so that no change, or a conservative change, is made in the amino acid coding sequence found at that position.

Preferably a patient sequence acceptor site is located within a relatively conserved region of the viral genome to facilitate introduction of the patient-derived segments. Alternatively, a patient sequence acceptor site is located between functionally important genes or regulatory sequences. A patient sequence acceptor site also can be located in or near regions in the viral genome that are relatively conserved to permit priming by the primer used to introduce the corresponding restriction site into the patient-derived segment.

To improve the representation of patient-derived segments further, pools of related primers accomodating viral sequence heterogeneity, or incorporating residues such as deoxyinosine which have multiple base-pairing capabilities, may be used (see e.g., Sambrook et al., 1989, Molecular Cloning: A Laboratory Manual, Cold Spring Harbor Laboratory, NY and Ausubel et al., 2002, Current Protocols in Molecular Biology, Greene Publishing Associates and Wiley Interscience, N.Y.). Sets of resistance test vectors having patient sequence acceptor sites that define the same or overlapping restriction site intervals may be used together in the drug susceptibility tests to provide representation of patient-derived segments that comprise internal restriction sites identical to a given patient sequence acceptor site, and would thus be under-represented in either resistance test vector alone.

5.3.3 Viral Segment and Viral Sequence

A viral segment is a viral sequence or viral gene encoding a gene product (e.g., a protein) that is the target of an anti-viral drug.

A functional viral sequence is a nucleic acid sequence (DNA or RNA) with functional activity, e.g., an open reading frame, an enhancer, promoter, polyadenylation site, site of action of a trans-acting factor, internal ribosome entry site (IRES), translation frameshift site, packaging sequence, integration sequence, splicing sequence etc. Examples of functional HCV sequences include, for example, the IRES and all or part of the polyprotein (e.g., C, E1, E2, NS2, NS3, NS4A, NS4B, NS5A and NS5B) (FIG. 1). For a drug that targets more than one functional viral sequence or viral gene product, patient-derived segments corresponding to each said viral gene are inserted in the resistance test vector. In the case of combination therapy where two or more anti-virals targeting two different functional viral sequences or viral gene products are being evaluated, patient-derived segments corresponding to each functional viral sequence or viral gene product are inserted into the resistance test vector, or into separate resistance test vectors. The patient-derived segments are inserted into a patient sequence acceptor site in the indicator gene viral vector, or, for example, a packaging vector, depending on the particular construction being used, as described herein.

5.3.4 Indicator Gene

An indicator or indicator gene is a nucleic acid encoding a protein, DNA or RNA structure that either directly or through a reaction gives rise to a measurable or noticeable aspect, e.g., a color or light of a measurable wavelength or in the case of DNA or RNA used as an indicator a change or generation of a specific DNA or RNA structure. See Schenbom et al., 1999, Mol Biotechnol. 13(1):29-44; Alam et al., 1990, Anal Biochem. 188(2):245-54. Examples of indicator genes include, for example, E. coli lacZ gene which encodes beta-galactosidase (Olesen et al., 1997, Methods Mol Biol. 63:61-70), the luc gene which encodes luciferase from, for example, Photonis pyralis (the firefly) (Gould et al., 1988, Anal Biochem. 175(1):5-13) or Renilla reniformis (the sea pansy) (Lorenz et al., 1991, Proc Natl Acad Sci U S A 88(10):4438-42), the E. coli phoA gene which encodes alkaline phosphatase (Yang et al., 1997, Biotechniques 23(6):1110-14), green fluorescent protein (Chalfie, 1995, Photochem Photobiol. 62(4):651-656), the bacterial CAT gene which encodes chloramphenicol acetyltransferase (Davey et al., 1995, Methods Mol Biol. 49:143-8), and the bacterial β-lactamase gene (Moore et al., 1997, Anal Biochem. 247(2):203-9; Zlokarnik, 2000, Methods Enzymol. 326:221-44).

Additional examples of indicator genes are secreted proteins or cell surface proteins that are readily measured by assay, such as radioimmunoassay (RIA), or fluorescent activated cell sorting (FACS), including, for example, growth factors, cytokines and cell surface antigens (e.g., growth hormone, II-2 or CD4, respectively). The term indicator gene also includes a selection gene, also referred to as a selectable marker. Examples of suitable selectable markers for mammalian cells include, but are not limited to, dihydrofolate reductase (DHFR), thymidine kinase or E. coli gpt or genes that code for resistance to the antibiotics hygromycin, neomycin, puromycin or zeocin. In the foregoing examples of indicator genes, the indicator gene and the patient-derived segment are discrete, i.e. distinct and separate genes.

Functional Indicator Gene

In one aspect, the invention provides a functional indicator gene. A functional gene is expressed in a packaging host cell or resistance test vector host cell independent of the patient-derived segment. However, a functional indicator gene cannot be expressed in a target host cell without the production of functional resistance test vector particles and their effective infection of the target host cell. In one embodiment, the indicator gene cassette, comprising control elements and a gene encoding an indicator protein, is inserted into the indicator gene viral vector, or packaging viral vector, with the same or opposite orientation as the native or foreign control element of the viral vector. In one embodiment, an indicator gene and its regulatory element (for example, the EMCV IRES) are placed in the same or opposite orientation as the HCV IRES, respectively.

Non-Functional Indicator Gene

In another aspect, the invention provides a non-functional indicator gene. A non-functional indicator gene is one that is not efficiently expressed in a packaging host cell transfected with the resistance test vector, which is then referred to a resistance test vector host cell, until it is converted into a functional indicator gene through the action of one or more of the patient-derived segment products. An indicator gene can be rendered non-functional through genetic manipulation according to this invention.

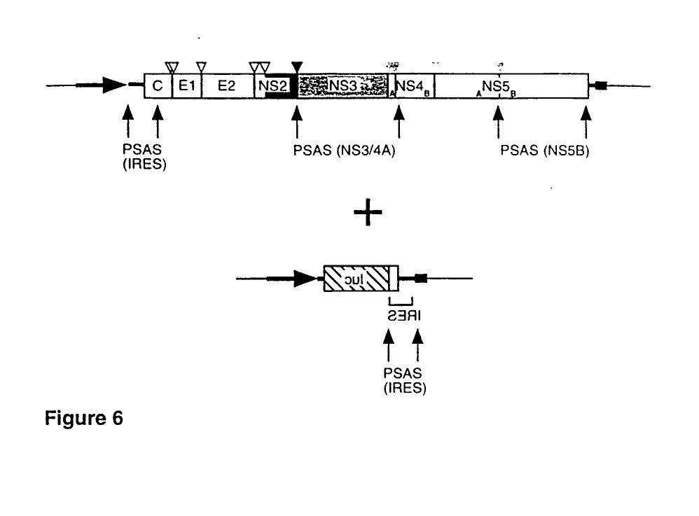

Antisense RNA Coding Region: In another embodiment, an indicator gene is rendered non-functional in transfected cells because its non-coding strand is transfected or expressed in transfected cells. In one embodiment, the indicator gene is non-functional until acted upon by the viral replication machinery. In a more particularly defined embodiment, the minigenome is introduced into the cells as antisense RNA, i.e., as a replicative intermediate RNA copy of the minigenome described above. For example, FIG. 6 presents a diagrammatic representation of the two part resistance test vector pXHCV-ASIRESluc comprising an antisense RNA mini-genome. The two constructs are co-transfected into cells. HCV non-structural proteins expressed from pXHCV act on both RNAs, leading to their replication. Expression in the transfected cells is dependent on the activity of NS5B, production of which is dependent in turn on the action of NS3/4A and the cis-acting regulatory elements such as the IRES. Thus the indicator gene is non-functional until acted upon by the viral replication machinery.

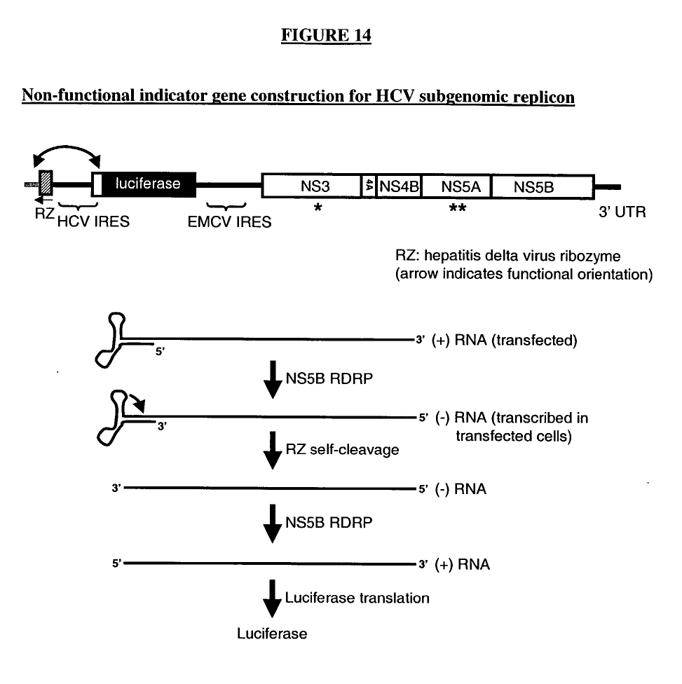

In another embodiment, the non-functional indicator gene is present on a replicon comprising a sequence that inhibits or prevents expression of the indicator gene on the input RNA, but which is removed by a replication-dependent mechanism, thus allowing greater expression of the indicator gene. In a more particularly defined embodiment, the replicon has at its 5′ end (1) a sequence that is complementary to a downstream sequence in the HCV IRES or in the indicator gene coding region such that the complementary sequences hybridize to each other and disrupt HCV IRES function, and (2) an antisense (and thus inactive) copy of the HDV ribozyme (RZ) (e.g., see Perotta et al., 1991, Nature 350:434-36; Chowrira et al, 1994, J Biol. Chem. 269: 25864; Wadkins et al., 2002, Cell Mol. Life Sci. 59:112-25). An example of this embodiment is shown in FIG. 14. The complementary sequence inhibits translation of the indicator gene (luciferase in FIG. 14) from the input RNA. However, the HCV NS proteins on the replicon are translated via another IRES. Replication of the input RNA strand produces an RNA having the sense strand of the RZ, which then cleaves itself from the RNA. Copies of this self-cleaved RNA do not have the complementary sequence, and so the HCV IRES is active and the luciferase gene is expressed.

5.3.5 Indicator Gene Viral Vector and Their Construction

In another aspect, the invention provides an indicator gene viral vector (“IGVV”). An IGVV is one or more vectors comprising an indicator gene and its control elements and one or more viral genes. The indicator gene viral vector can be assembled from an indicator gene cassette and a viral vector. The IGVV can optionally include an enhancer, splicing signals, polyadenylation sequences, transcriptional terminators, or other regulatory sequences in addition to the indicator gene cassette and the viral vector. The IGVV can be functional or nonfunctional. In the event that the viral segments which are the target of the anti-viral drug (e.g., patient derived segments) are not included in the IGVV they can be provided in a second vector, which can be a packaging viral vector.

An indicator gene cassette comprises an indicator gene and, optionally, control elements. A viral vector is a vector comprising some or all of the following: viral genes encoding a gene product, control sequences, viral packaging sequences. The viral vector can additionally include one or more viral segments one or more of which can be the target of an anti-viral drug. Examples of a viral vector that comprise viral genes include, but are not limited to, a genomic viral vector and a subgenomic viral vector. A genomic viral vector is a vector that can comprise a deletion of one or more viral genes to render the virus replication incompetent, but which otherwise preserves the mRNA expression and processing characteristics of the complete virus. In one embodiment for an HCV drug susceptibility test, the genomic viral vector comprises C, E1, E2, NS2, NS3, NS4, and NS5. A subgenomic viral vector is a vector that can comprise the coding region of one or more viral genes which can encode the protein(s) that are the target(s) of the anti-viral drug. For HCV, a preferred embodiment is a subgenomic viral vector comprising the HCV NS2, NS3, NS4, NS5 genes.

5.3.6 Host Cells

In another aspect, the invention provides host cells that can be of several different types and into which resistance test vectors or other sequences are introduced. The component of the resistance test vector system that comprises the indicator gene can be delivered to the target host cell at the time of infection or can be stably integrated into the target host cell chromosomal DNA.

Host cells used in the susceptibility assays of the invention can be any suitable host cells known in the art. Examples of host cells include, but are not limited to, mammalian cells such as human cells, for example, hepatocytes, hepatoma cell lines or other cells. In a preferred embodiment, host cells are derived from human tissues and cells which are the principle targets of viral infection.

Host cells can be of various types, including, but not limited to, packaging host cells, resistance test vector host cells, or target host cells. A packaging host cell is a host cell that provides the trans-acting factors and viral packaging proteins required by the replication defective viral vectors used herein, such as the resistance test vectors, to produce resistance test vector viral particles. The packaging proteins can be provided for by the expression of viral genes present within the resistance test vector itself, a packaging expression vector(s), or both. A packaging host cell is a host cell that can be transfected with one or more packaging expression vectors. A host cell transfected with a resistance test vector is referred to herein as a resistance test vector host cell or a packaging host cell/resistance test vector host cell. Host cells that can be used as packaging host cells for HCV include, but are not limited to, HepG2 (Hiramatsu et al., 1997, J. Viral Hepatol., 4(suppl.1):61-67), Huh7 (Yoo et al., 1995, J. Virol. 69, 32-38), Vero (Valli et al., 1997, Res. Virol. 148:181-186), Molt4Ma (Shimizu et al., 1992, PNAS 89:5477-5481), HPBMa (Shimizu et al., 1993, PNAS 90:6037-6041; Shimizu and Yoshikura, 1994, J. Virol. 68, 8406-8408; Shimizu et al., 1994, J. Virol. 68:1494-1500), MT-2 (Mizutani et al., 1996, J. Virol. 70:7219-7223), or human hepatoma cell lines, e.g., Hep3B, PLC/PRF/5, Huh6, HLE, SK-Hep1, HepT1, HepT3 and HLF (Hoshida et al., 2001, Genome Informatics 12:257-58; Kawai et al, 2001, Hepatology 33:676-91; Pietsch, et al., 1996, Lab Invest 74:809-18).

A target host cell is a cell that is infected by resistance test vector viral particles produced by the resistance test vector host cell in which expression or inhibition of the indicator gene takes place. Host cells that can be used as target host cells for HCV include, but are not limited to, HepG2 (Hiramatsu et al., 1997, J. Viral Hepatol., 4(suppl.1):61-67), Huh7 (Yoo et al., 1995, J. Virol. 69, 32-38), Vero (Valli et al., 1997, Res. Virol. 148:181-186), Molt4Ma (Shimizu et al., 1992, PNAS 89:5477-5481), HPBMa (Shimizu et al., 1993, PNAS 90:6037-6041; Shimizu and Yoshikura, 1994, J. Virol. 68, 8406-8408; Shimizu et al., 1994, J. Virol. 68:1494-1500), MT-2 (Mizutani et al., 1996, J. Virol. 70:7219-7223), or human hepatoma cell lines, e.g., Hep3B, PLC/PRF/5, Huh6, HLE, SK-Hep1, HepT1, HepT3 and HLF (Hoshida et al., 2001, Genome Informatics 12:257-58; Kawai et al., 2001, Hepatology 33:676-91; Pietsch, et al., 1996, Lab Invest 74:809-18).

5.4 Drug Susceptibility Tests

The present invention provides methods for determining the susceptibility of a pathogenic flavivirus to anti-viral compounds. The drug susceptibility (resistance and sensitivity) tests of this invention can be carried out in one or more host cells. Viral drug susceptibility can be determined as the concentration of the anti-viral agent at which a given percentage of indicator gene expression is inhibited (e.g., IC₅₀ or IC₉₀). A standard curve for drug susceptibility of a given anti-viral drug can be developed for a viral segment that is either a standard laboratory viral segment or from a drug-naive patient (i.e., a patient who has not received any anti-viral drug) using the method of this invention. Correspondingly, viral drug resistance is a decrease in viral drug susceptibility for a given patient measured either by comparing the drug susceptibility to such a given standard or by making sequential measurement in the same patient over time, as determined by decreased inhibition of indicator gene expression (i.e., increased indicator gene expression in the presence of drug).

In one aspect, the present invention provides a one cell susceptibility assay. In a one cell susceptibility test, a host cell is transfected with the resistance test vector and the appropriate packaging expression vector(s) to produce a resistance test vector host cell. Individual anti-viral agents, or combinations thereof, can be added to individual plates of transfected cells at the time of their transfection, or at another appropriate time, at an appropriate range of concentrations known to one of skill in the art. At an appropriate time after transfection, cells can be collected and assayed for indicator gene expression as described herein. Transfected cells in the culture as well as superinfected cells in the culture can serve as target host cells for indicator gene expression. The reduction in luciferase activity observed for cells transfected in the presence of a given anti-viral agent, or agents as compared to a control run in the absence of the anti-viral agent(s) can be used to calculate the apparent inhibitory concentration of the agent, or combination of agents, for the viral target product encoded by the patient-derived segments present in the resistance test vector as described herein.

In another aspect of the drug susceptibility test, a single host cell (the resistance test vector host cell) can also serve as a target host cell. The packaging host cells are transfected and produce resistance test vector viral particles and some of the packaging host cells can also become the target of infection by the resistance test vector particles. Drug susceptibility tests employing a single host cell type are preferably performed with viral resistance test vectors comprising a non-functional indicator gene with an antisense strand indicator RNA. Such indicator genes are not efficiently expressed upon transfection of a first cell, but are only efficiently expressed upon infection of a second cell and thus provide an opportunity to measure the effect of the anti-viral agent under evaluation.

In another aspect, the present invention provides a two cell susceptibility assay. In one embodiment of a two cell susceptibility assay, resistance test vector viral particles are produced by a first host cell (the resistance test vector host cell) that is prepared by transfecting a packaging host cell with the resistance test vector and packaging expression vector(s). The resistance test vector viral particles are then used to infect a second host cell (the target host cell) in which the expression of the indicator gene is measured. A two cell susceptibility assay can be performed using either a functional or non-functional indicator gene. Functional indicator genes are efficiently expressed upon transfection of the packaging host cell and would require infection of a target host cell with resistance test vector host cell supernatant to carry out the test of this invention. Non-functional indicator genes with an antisense strand indicator RNA are not efficiently expressed upon transfection of the packaging host cell and thus the infection of the target host cell can be achieved either by co-cultivation by the resistance test vector host cell and the target host cell or through infection of the target host cell using the resistance test vector host cell supernatant. In one embodiment, the resistance test vector comprises a functional indicator gene that uses a two cell system using filtered supernatants from the resistance test vector host cells to infect the target host cell.

Particle-based resistance tests can be carried out with resistance test vectors derived from genomic viral vectors that include, but are not limited to, pXHCV-luc, pXHCV-IRESluc, pXHCV/pXIRESluc, pXHCV/pXASIRESluc; pXluc-NSHCV/pXsHCV, pXBVDV(HCVNS3)luc, and pXBVDV(HCVNS5B)Iuc. Alternatively, particle-based resistance tests can be carried out with similar resistance test vectors but wherein the indicator gene is on a separate nucleic acid, e.g., on an extrachromosomal element or integrated into a host cell's genome.

A two cell susceptibility assay can be performed by transfecting the packaging host cells with the resistance test vector and appropriate packaging expression vector(s) to produce resistance test vector host cells. Individual anti-viral agents for the flavivirus (e.g., HCV), including the protease inhibitors, IRES inhibitors, and the polymerase inhibitors as well as combinations thereof, can be added to individual plates of packaging host cells at the time of their transfection, at an appropriate range of concentrations which would be known to one of skill in the art. At an appropriate time after transfection, for example, 24-48 hours after transfection, target host cells can be infected by co-cultivation with resistance test vector host cells or with resistance test vector viral particles obtained from filtered supernatants of resistance test vector host cells. Each anti-viral agent, or combination thereof, can be added to the target host cells prior to or at the time of infection to achieve the same final concentration of the given agent, or agents, present during the transfection.

The expression or inhibition of the indicator gene in the target host cells infected by co-cultivation or with filtered viral supernatants can be determined by assay of indicator gene expression, for example, if the indicator gene is the firefly luc gene, by measuring luciferase activity. The reduction in luciferase activity observed for target host cells infected with a given preparation of resistance test vector viral particles in the presence of a particular anti-viral agent, or agents, as compared to a control run in the absence of the anti-viral agent, generally relates to the log of the concentration of the anti-viral agent as a sigmoidal curve. This inhibition curve can be used to calculate the apparent inhibitory concentration of that agent, or combination of agents, for the viral target product encoded by the patient-derived segments present in the resistance test vector.

5.5 Anti-viral Drugs and Drug Candidates

Any anti-viral drug known in the art can be used with the susceptibility assays of the invention. Examples of anti-viral agents that can be used include, but are not limited to, those that target flaviviral proteins (e.g., protease/helicase, RDRP) or nucleic acids, or that otherwise inhibit any process or aspect of the viral life cycle, including, e.g., binding of or entry into cells, genome replication or processing, protein translation, modification or processing, proteolysis, capsid assembly, or viral maturation. Preferably the flavivirus is HCV. In one embodiment, the anti-viral agent targets viral NS3 protein. In another embodiment, the anti-viral agent targets viral NS5B. In yet another embodiment, the anti-viral agent is an anti-sense nucleic acid. In still another embodiment, the anti-viral agent is a ribozyme that targets specific sequences in the viral RNA.

An anti-viral drug or compound is said to “target” an HCV nucleic acid or protein if the drug or compound exerts its anti-viral effects directly or indirectly on the nucleic acid or protein. For example, without being bound to any particular theory, some studies have shown that interferon's complex anti-viral effect is mediated by a short sequence in the HCV NS5A protein termed the interferon sensitivity determining region (ISDR; Witherell et al., 2001, J. Med. Virol. 63:8-16), suggesting that the ISDR is a target of interferon. An anti-viral drug or compound can directly or indirectly target an HCV nucleic acid or protein. For example, without being bound to any particular theory, the anti-viral compound ribavirin, although it is a nucleoside analog, is thought to not exert its anti-HCV effects through direct contact with or incorporation by the HCV NS5B polymerase (Pawlotsky, 2000, Hepatology 32:889-96). Thus, it might indirectly inhibit HCV through a direct interaction with one or more host cell factors.

The anti-viral agent or agents can be added to the assay at any appropriate time or stage. One of skill in the art would know the appropriate time or stage to add the anti-viral agent or agents depending on the target of the agent or agents. In one embodiment, the anti-viral drugs are present throughout the assay In another embodiment, agent or agents are added to individual plates of packaging host cells at the time of their transfection with a resistance test vector, at an appropriate range of concentrations, known to one of skill in the art. In yet another embodiment, the agents are added to the target host cells at the time of infection to achieve the same final concentration added during transfections. The test concentration is selected from a range of concentrations designed to give a satisfactory inhibition profile for resistant and sensitive isolates, and is known to one of skill in the art, or can be determined by one of skill in the art using the compositions and methods provided herein.

In one aspect, the invention provides a method for assaying the antiviral activity of a candidate anti-viral compound. The candidate anti-viral compound can be added to the test system at an appropriate concentration and at selected times depending upon the protein target of the candidate anti-viral as described herein. In one embodiment, more than one candidate anti-viral compound is tested in combination with an approved anti-viral drug or a compound which is undergoing clinical trials. The effectiveness of the candidate anti-viral can be evaluated by measuring the expression or inhibition of the indicator gene as described herein.

In yet another aspect, the present invention also provides a method for screening for viral mutants that are resistant to an anti-viral compound. In one embodiment, an HCV-derived nucleic acid from a virus comprising one or more mutations is used in the drug susceptability assays of the invention, and, if the HCV-derived nucleic acid has a decreased susceptability for the anti-viral compound, then the is collected or analyzed to determine one or more of its mutations. A library of viral mutants having resistance to anti-viral compounds can thus be assembled enabling the screening of candidate anti-virals, alone or in combination, using the drug susceptibility assay as described herein. This will enable one of ordinary skill to identify effective anti-virals and design effective therapeutic regimens.

5.6 General Materials and Methods

Most of the techniques used to construct vectors, and transfect and infect cells, are widely practiced in the art, and most practitioners are familiar with the standard resource materials which describe specific conditions and procedures. However, for convenience, the following paragraphs may serve as a guideline.

Plasmids and vectors for cloning are designated by a lower case p followed by letters and/or numbers. The starting plasmids herein are either commercially available, publicly available on an unrestricted basis, or can be constructed from available plasmids in accord with published procedures. In addition, plasmids equivalent to those described are known in the art and will be apparent to one of skill in the art.

Construction of the vectors of the invention employs standard ligation and restriction techniques which are well understood in the art (see Ausubel et al., 2002, Current Protocols in Molecular Biology, Wiley—Interscience or Sambrook et al., 1989, Molecular Cloning: A laboratory Manual, Cold Spring Harbor Laboratory, N.Y.). Isolated plasmids, DNA sequences, or synthesized oligonucleotides can be cleaved, tailored, and re-ligated in the form desired. The sequences of DNA constructs incorporating synthetic DNA can be confirmed by DNA sequence analysis (see, e.g., Sanger et al., 1977 Proc. Natl. Acad. Sci. 74, 5463-5467).

Digestion of DNA refers to catalytic cleavage of the DNA with a restriction enzyme. Various restriction enzymes can be used and those used herein are commercially available and their reaction conditions, co-factors and other requirements are known to one of skill in the art. For analytical purposes, typically 1 μg of plasmid or DNA fragment can be used with about 2 units of enzyme in about 20 μl of buffer solution. Alternatively, an excess of restriction enzyme can be used to insure complete digestion of the DNA substrate. Incubation times of about one hour to about two hours at about 37° C. are workable, although variations can be tolerated. After each incubation, protein can be removed by extraction with phenol/chloroform, and can be followed by ether extraction, and the nucleic acid recovered from aqueous fractions by precipitation with ethanol. Optionally, size separation of the cleaved fragments can be performed by polyacrylamide gel or agarose gel electrophoresis using standard techniques. A general description of size separation is found in Methods of Enzymology 1980, 65:499-560.

Restriction cleaved fragments can be blunt ended by treating with the large fragment of E coli DNA polymerase I (Klenow) in the presence of the four deoxynucleotide triphosphates (dNTPs) using incubation times of about 15 to 25 minutes at about 20° C. in approximately 50 mM Tris (pH 7.6) 50 mM NaCl, 6 mM MgCl₂, 6 mM DTT and about 5-10 micromole dNTPs. The Klenow fragment fills in at 5′ sticky ends but hydrolyses protruding 3′ single strands, even though the four dNTPs are present. Optionally, selective repair can be performed by supplying only one of the dNTPs, or with selected dNTPs, within the limitations dictated by the nature of the sticky ends. After treatment with Klenow, the mixture can be extracted with phenol/chloroform and ethanol precipitated. Treatment under appropriate conditions with S1 nuclease or Bal-31 can result in hydrolysis of any single-stranded portion.

Ligations can be performed in 15-50 μl volumes under standard conditions and temperatures, for example, 20 mM Tris-Cl pH 7.5, 10 mM MgCl2, 10 mM DTT, 33 mg/ml BSA, 10 mM50 mM NaCl, and either 40 μM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 33-100 μg/ml total DNA concentrations (5-100 mM total end concentration). Intermolecular blunt end ligations (usually employing a 10-30 fold molar excess of linkers) can be performed at 1 μM total ends concentration.

Transient expression refers to unamplified expression within about one day to two weeks of transfection. The optimal time for transient expression of a particular desired heterologous protein can vary depending on several factors including, for example, any trans-acting factors which may be employed, translational control mechanisms and the host cell. Without being bound by theory, it is believed that transient expression occurs when the particular plasmid that has been transfected functions, i.e., is transcribed and translated During this time the plasmid DNA which has entered the cell is typically transferred to the nucleus and is present in a nonintegrated state, free within the nucleus. Transcription of the plasmid taken up by the cell can occur during this period. Following transfection the plasmid DNA may become degraded or diluted by cell division. Random integration within the cell chromatin can occur.

Vectors comprising promoters and control sequences derived from species compatible with the host cell can be used with a particular host cell. Promoters suitable for use with prokaryotic hosts include, but are not limited to, the beta-lactamase and lactose promoter systems, alkaline phosphatase, the tryptophan (trp) promoter system and hybrid promoters such as tac promoter. However, other functional bacterial promoters can also be used. In addition to prokaryotes, eukaryotic microbes such as yeast cultures can also be used. Saccharomyces cerevisiae, or common baker's yeast is the most commonly used eukaryotic microorganism, although a number of other strains are commonly available. Promoters controlling transcription from vectors in mammalian host cells can be obtained from various sources, for example, the genomes of viruses such as polyoma, simian virus 40 (SV40), adenovirus, retroviruses, hepatitis B virus and cytomegalovirus, or from heterologous mammalian promoters, e.g., β-actin promoter. The early and late promoters of the SV40 virus are conveniently obtained as an SV40 restriction fragment which also comprise the SV40 viral origin of replication. The immediate early promoter of the human cytomegalovirus is conveniently obtained as a HindIII E restriction fragment. Promoters from the host cell or related species can also be are used.

The vectors used herein can comprise a selection gene, also termed a selectable marker. A selection gene can encodes a protein necessary for the survival or growth of a host cell transformed with the vector. Examples of selectable markers for mammalian cells include, but, are not limited to, the dihydrofolate reductase gene (DHFR), the omithine decarboxylase gene, the multi-drug resistance gene (mdr), the adenosine deaminase gene, and the glutamine synthase gene. Without wishing to be bound by theory, when selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. The second category is referred to as dominant selection which refers to a selection scheme used in any cell type and does not require the use of a mutant cell line. These schemes typically use a drug to arrest growth of a host cell. Those cells which have a gene that can express a protein that conveys resistance to the drug can survive the selection. Examples of drugs that can be used for selection include, but, are not limited to, neomycin (G418 or genticin) (Southern and Berg, 1982, J. Molec. Appl. Genet. 1, 327), mycophenolic acid (xgpt) (Mulligan and Berg, 1980, Science 209, 1422), or hygromycin (Sugden et al., 1985, Mol. Cell. Biol. 5, 410-413). Bacterial genes under eukaryotic control can be used to convey resistance to these drugs.

Transfection is the introduction of nucleic acid, (e.g., DNA) into a host cell so that the nucleic acid is expressed, either functionally or otherwise. The nucleic acid can also replicate either as an extrachromosomal element or by chromosomal integration. Various methods, known to one of skill in the art, can be used for transfection of the host cells. An example of such a method includes, but, is not limited to, the calcium phosphate co-precipitation method of Graham and van der Eb, 1973, Virology 52:456-457. Alternative methods for transfection are electroporation, the DEAE-dextran method, lipofection and biolistics (Kriegler, 1990, Gene Transfer and Expression: A Laboratory Manual, Stockton Press).

Host cells can be transfected with the expression vectors of the present invention and cultured in conventional nutrient media modified as appropriate for inducing promoters, selecting transformants or amplifying genes. Host cells can be cultured in F12:DMEM (Gibco) 50:50 with 1% FBS and added glutamine and without antibiotics. The culture conditions, such as temperature, pH and the like, are those previously used with the host cell selected for expression, and will be apparent to one of skill in the art.

5.7 Phenotypic Drug Susceptibility Assays

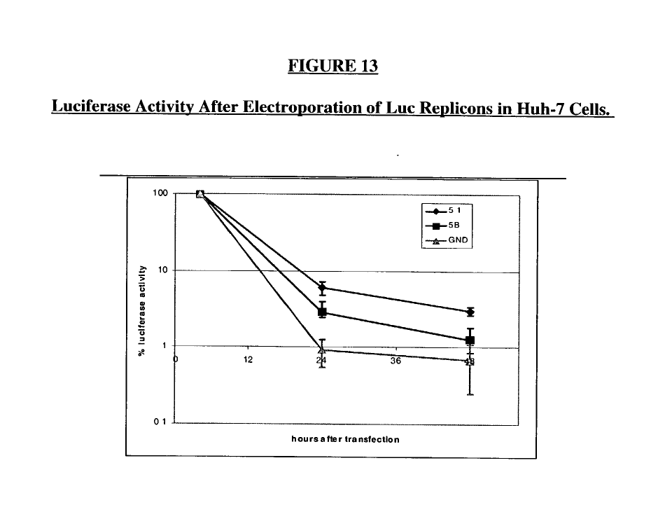

In one aspect, the present invention provides a phenotypic assay for determining the susceptibility of a pathogenic flavivirus to anti-viral agents. In one embodiment, the phentotypic drug susceptability assay is used to tailor an antiviral drug therapy to a patient infected with a flavivirus, e.g., HCV. A phenotypic assay can be performed using recombinant viral replicons that comprise an indicator gene. A replicon is a nucleic acid comprising one or more HCV-derived segments that allow the nucleic acid to replicate in an appropriate host cell. Examples of replicons are provided in FIGS. 12 and 14. Examples of indicator genes are provided above. A segment of a viral replicon, for example, an HCV replicon, that encodes a specific anti-viral drug target can be derived from patient isolates. Examples of targets of anti-viral agents include, but are not limited to, HCV protease/helicase (NS3) and RNA dependent RNA polymerase (NS5B), and other viral segments and functional viral sequences discussed above. Various host cells, examples of which are provided above, can be transfected with the viral replicon and drug susceptibility can be evaluated by comparing indicator gene expression levels in the presence and absence of anti-viral drugs as described herein.

Convenient restriction sites to accommodate the insertion of patient-derived sequences can be introduced into a working viral (for example, HCV) replicon system. Alternative indicator genes and control elements can also be introduced into the working viral replicon system.

The amplification (e.g., by RT-PCR) of the viral drug target sequences from plasma, serum, or other specimens, and their incorporation into the replicon vectors can be optimized. In addition, methods for viral replicon RNA transcription and transfection can be optimized.

Various cell lines, including those described above, can be evaluated for their ability to support viral replicon replication, and the assay formats provided herein can be modified to improve the results obtained with a particular cell line.

This assay is useful, inter alia, for measuring dose-dependent inhibition of viral replicon replication using candidate anti-viral drugs or detecting changes in viral drug susceptibility conferred by verified drug resistance mutations or viral sequences derived from patients treated with investigational drugs.

In one embodiment, the assay is used for selecting optimal drug combinations for patients considering, undergoing, or failing anti-viral drug therapy. In another embodiment, the assay is used for monitoring the development of drug resistant virus during the clinical evaluation of investigational anti-viral agents. In yet another embodiment, the assay is used to aid the discovery and development of second-generation drugs that are active against drug resistant strains by screening new drug candidates against libraries of viral replicon vectors comprising mutations. In a more particularly defined embodiment, the viral replicon vectors are derived from patient samples. The phenotypic drug susceptibility assay offers several significant advantages over the existing viral replicon technology including, reproducible amplification of patient viral sequences from diverse subtypes over a wide range of viral loads, reliable methods for the construction of resistance test vectors comprising the patient sequences, and the ability to measure drug susceptibility in the context of a virus-like genome.

In one embodiment, a reporter gene replicon-based assay that determines the activity of viral inhibitors, such as, HCV NS3 helicase/protease or NS5B polymerase inhibitors, and changes in susceptibility of patient viruses to viral inhibitors can be performed as follows.

The NS3 or NS5B region of HCV from patient plasma is amplified by RT-PCR. Resistance test vectors are generated by inserting the amplified patient-derived sequence into a replicon vector using restriction enzymes with recognition sites in the PCR primers and the appropriate positions in the replicon vector. In a more particularly defined embodiment, different resistance test vectors (“RTVs”) comprising different sets of adaptive mutations are used in the assay, depending on the target patient sequence. An adaptive mutation as used herein is a mutation, typically in an HCV-derived nucleic acid or protein, that is associated with an improvement in the functioning of an assay of the invention, e.g., by increasing the rate of replication of a replicon used in the assay. Examples of adaptive mutations are provided below.

In vitro transcription of the recombinant RTVs to generate positive (+) sense replicon RNA for transfection is performed. This is followed by transfection of host cells, for example, Huh7 cells, with the in vitro transcribed replicon RNA and measurement of reporter gene (for example, luciferase) activity shortly (e.g., 4 hours) after transfection.

NS3 or NS5B inhibitor is added to the transfected cells, either before or after transfection, as appropriate. Reporter gene activity is measured after expression from the input RNA has faded to background levels. This can be determined by, for example, using a control RNA containing a mutation that greatly reduces or inactivates the activity of the RDRP. Reporter gene expression at later times is dependent on viral NS3 and NS5B gene activity, assuming that input transfected RNA is unstable, that RNA replication cannot occur unless NS5B is correctly processed from the polyprotein, and that NS3 helicase and protease function is required for RNA replication and NS5B processing, respectively.

Lastly, the IC₅₀ based on a plot of percent inhibition of reporter gene activity vs. log10 of drug concentration is calculated.

5.8 Drug Susceptibility Tests and Drug Screening

5.8.1 HCV Drug Susceptibility Test Using RTVs Comprising Patient-Derived Segment(s) and a Functional Indicator Gene Fused to the HCV Polyprotein

In one aspect, the present invention provides an assay for determining the susceptibility of HCV to anti-viral agents using RTVs comprising patient-derived segment(s) and an indicator gene fused to the HCV polyprotein. The assay optionally includes construction of an IGVV, and/or construction of a RTV.

Construction of the Indicator Gene Viral Vector

An indicator gene viral vector (IGVV) can be designed using HCV genomic viral vectors comprising an indicator gene fused to the HCV polyprotein. The IGVV can be constructed by inserting an open reading frame for the indicator gene in a cDNA construct comprising the entire HCV genome producing an in-frame fusion protein. The IGVV can optionally comprise all the cis-acting regulatory elements in the 5′ and 3′ untranslated regions (UTRs) required for replication, transcription, and translation of the HCV RNA.

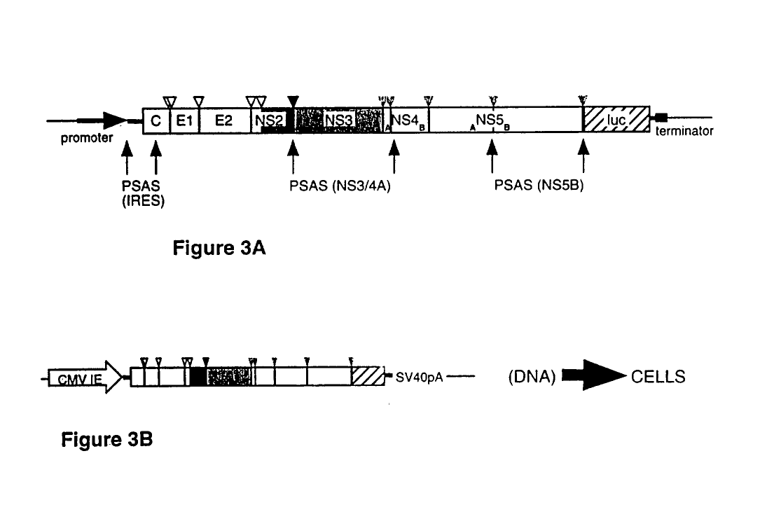

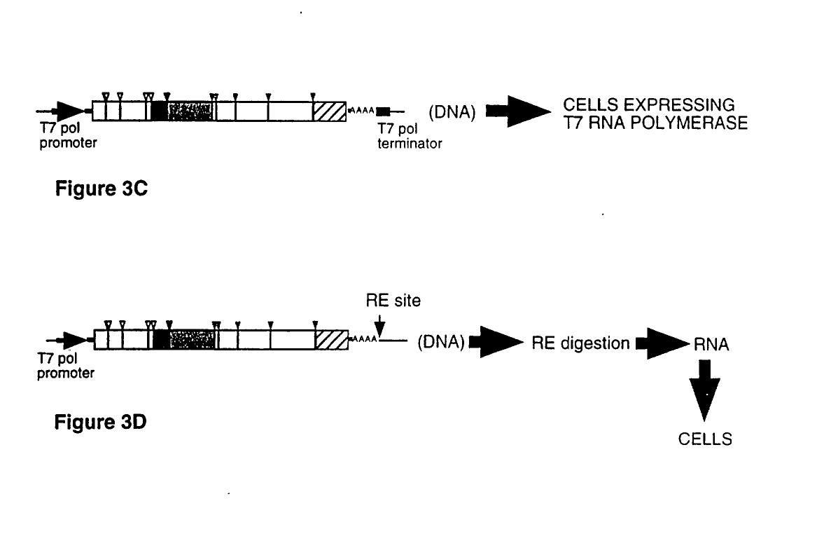

In one embodiment, the luciferase open reading frame is placed immediately downstream of the NS5 coding region, with a spacer region comprising the recognition sequence for the NS3/4A protease (FIG. 3A). FIG. 3A is a diagrammatic representation of the resistance test vector (pXHCV-luc, where X is either CMV or T7), with patient sequence acceptor sites for transfer of patient derived segments indicated by arrows below the polyprotein (PSAS). The promoter and terminator sequences are indicated generically in this figure as well as in subsequent figures, as several different types of regulatory elements may be used. The luciferase reporter gene is expressed as a fusion protein with the HCV polyprotein and then cleaved off by the action of NS3/4A. An example of such a cleavage site is TEDVVCC-SMSYTWT, representing the junction between NS5A and NS5B (Grakoui et al 1993, J. Virol. 67:2832; Steinkühler et aL, 1996, J. Virol. 70:6694). The expected cleavage products are HCV NS5B comprising a C-terminal extension (e.g., TEDVVCC), and luciferase comprising an N-terminal extension (e.g., SMSYTWT).

In another embodiment, the luciferase open reading frame is placed between the NS5A and NS5B open reading frames, with an NS5A-5B cleavage sequence at both the N-terminal NS5A-luc and C-terminal luc-NS5B junctions. The luciferase protein produced from this construct comprises an N-terminal SMSYTWT and a C-terminal TEDVVCC extension.

In yet another embodiment, the luciferase open reading frame is placed between the NS4B and NS5A open reading frames, with an NS4B-5A cleavage sequence (SECTTPC-SGSWLRD) at both the N-terminal NS4B-luc and C-terminal luc-NS5A junctions. The luciferase protein produced from this construct comprises an N-terminal SGSWLRD and a C-terminal SECTTPC extension.

In still another embodiment, the luciferase open reading frame is placed between the NS4A and NS4B open reading frames, with an NS4A-4B cleavage sequence (FDEMEEC-SQHLPYI) at both the N-terminal NS4A-luc and C-terminal luc-NS4B junctions. The luciferase protein produced from this construct comprises an N-terminal SQHLPYI and a C-terminal FDEMEEC extension.

The viral vector can be assembled from a full length cDNA construct of HCV, which consists of (in the 5′ to 3′ orientation) the 5′ UTR, the open reading frame for the 3010 amino acid polyprotein, and the 3′ UTR. The polyprotein can comprise a capsid (C) open reading frame, envelope glycoprotein genes (E1 and E2), NS2 (a cis-acting auto-protease that cleaves the polyprotein at a specific site at the NS2-NS3 junction), NS3 (helicase and serine protease), NS4A (required as a cofactor for NS3 activity), NS4B, NS5A, and NS5B (the RNA-dependent RNA polymerase) open reading frames. The luciferase open reading frame also can be present within the polyprotein open reading frame, located variously as described above.

In one embodiment, the IGVV comprises a eukaryotic promoter at the 5′ end of the HCV sequences for the production of RNA in transfected cells, and a transcription terminator at the 3′ end. Examples of transcription promoters include, but are not limited to, the CMV intermediate-early promoter, or the SV40 promoter; examples of transcription terminators include, but are not limited to, the transcription terminator/polyadenylation signals found in SV40 or the human β-globin gene (see FIG. 3B). FIG. 3B is a diagrammatic representation of a resistance test vector pCMVHCV-luc comprising the CMV IE promoter and SV40 polyadenylation signal. RNA is transcribed in the nucleus of cells into which the DNA of this vector is transfected by cellular RNA polymerases, then transported to the cytoplasm where translation and replication can occur.

In a second embodiment, the promoter is a promoter for bacteriophage RNA polymerases such as T7, T3, or SP6, and the terminator is a sequence signaling termination of transcription that is recognized by the polymerase, or a self-cleaving ribozyme (e.g., see Perotta et al., 1991, Nature 350:434-36; Chowrira et al., 1994, J. Biol. Chem. 269:25864; Wadkins et al., 2002, Cell Mol. Life Sci. 59:112-25). The IGVV is transfected as DNA into cells expressing the RNA polymerase in the cytoplasm. Such expression can be achieved by several methods including, but not limited to, cotransfection with a polymerase expression vector, infection with a recombinant vaccinia virus expressing the polymerase (Fuerst et al., 1986, PNAS 83:8122), and by previously establishing a cell line permanently expressing the polymerase (see FIG. 3C). In FIG. 3C, the DNA of a resistance test vector (pT7HCV-luc1) comprising the T7 RNA polymerase promoter and T7 RNA polymerase terminator is transfected into cells expressing T7 RNA polymerase (for example, after infection with recombinant vaccinia virus or by co-transfection with a T7 RNA polymerase expression plasmid). The RNA is transcribed in the cytoplasm by T7 polymerase. The IGVV of this embodiment can additionally comprise a functional 3′ end sequence, so that the transcribed RNA can comprise a functional 3′ end sequence.

In a third embodiment, the IGVV with a bacteriophage RNA polymerase promoter at the 5′ end and a terminator sequence at the 3′ end is transcribed in vitro and the nucleic acid representing the IGVV is transfected as RNA. The terminator can be a specific sequence recognized by the bacteriophage RNA polymerase as a termination site or a self-cleaving ribozyme (see Perotta et al., 1991, Nature 350:434-36; Chowrira et al., 1994, J. Biol. Chem. 269: 25864; Wadkins et al., 2002, Cell Mol. Life Sci. 59:112-25), or, the terminator can be a restriction endonuclease site allowing for linearization of the DNA template prior to transcription (see FIG. 3D). FIG. 3D shows a resistance test vector (pT7HCV-luc2) comprising the T7 RNA polymerase promoter and a restriction site placed at the 3′ end for linearization of the DNA prior to transcription in vitro. The synthetic RNA is then transfected directly into cells and translation and replication can occur. In this embodiment the vector also can comprise a functional 3′ end sequence.

In transfected cells, the RNA can be translated, using an internal initiation mechanism via the internal ribosome entry sequence (IRES), to yield the HCV polyprotein-luc fusion protein. Release of active luciferase from the HCV polyprotein fusion is dependent on the action of NS3/4A, itself expressed from the genomic RNA. High level expression can take place when the genomic RNA is replicated and amplified in the transfected cells, which is dependent on the action of the viral polymerase NS5B as well as the viral proteases NS2 and NS3/4A. In embodiments where luciferase is inactive when it is part of the large HCV polyprotein, activity can be measured directly in the transfected cells since release of active luciferase is dependent on HCV RNA replication (one cell assay). In embodiments where luciferase has significant activity as a fusion protein, progeny virions can be collected and used to infect new target cells (two cell assay). Transfer of the IGVV RNA from the transfected cells to the infected target cells is dependent on replication and encapsidation of the RNA in the transfected cells, which in turn is dependent on the correct expression, processing and activity of the HCV viral structural and non-structural proteins. To augment the efficiency of transfer (i.e., packaging of the IGVV RNA into new virions) the target cells can be simultaneously infected with wild-type HCV virus or transfected with wild type HCV RNA or cDNA expression constructs. To further augment the replication and packaging of the IGVV RNA, input RNAs (see FIG. 3D) can be co-transfected with purified NS5B protein (i.e., as RNP complexes), so that transcription can commence immediately upon uptake into the cells.

Construction of Resistance Test Vectors

A resistance test vector (RTV) is one or more nucleic acids which, taken together, comprise a patient-derived segment and an indicator gene. One or more nucleic acids comprising the vector can be integrated into the host cell's genome (e.g., the patient-derived segment can be present in an extrachromosomal nucleic acid (e.g., an IGVV) and the indicator gene integrated into a host cell's genome). An RTV comprising more than one nucleic acid is referred to herein as a “resistance test vector system” for purposes of clarity but is nevertheless understood to be a resistance test vector.

In one aspect, the invention provides a method of making an RTV by identifying a gene encoding a target of a drug in an IGVV, introducing into or identifying in the IGVV a unique patient sequence acceptor site, and replacing the target gene with a PDS.

In one embodiment, a region of the HCV genome (e.g., NS3/4A, NS5B, or the IRES) that is or encodes a target of an anti-viral agent is removed from the IGVV and replaced with the corresponding region derived from viruses and/or RNA present in the blood and/or cells of an infected patient (patient-derived segment, or PDS).

In a more specifically defined embodiment, the anti-viral agent is a NS3/4A protease inhibitor and the IGVV is modified by introducing a patient sequence acceptor site (“PSAS”), in or near the NS3/4A genes (nucleotides 3418-5473 of the H strain of HCV). The patient derived segment obtained from the patient derived virus is then transferred into the PSAS in the IGVV (FIG. 3A). The wild-type NS3/4A region is removed from the IGVV by digestion with restriction endonucleases recognizing the patient sequence acceptor sites. This region is then replaced with DNA fragments generated by RT/PCR from patient-derived viral RNA obtained from plasma or serum or cells. The PCR products are generated using primers which comprise the restriction endonuclease sites required for generation of compatible cohesive ends for cloning into the digested IGVV. RT and PCR primer binding sites are selected, and primer sequences designed, to enable amplification of as many different subtypes of HCV as possible.

In another more specifically defined embodiment, the anti-viral agent is an inhibitor of the NS5B RDRP and the sequences spanning the NS5B open reading frame (nucleotides 7601-9373 of the H strain of HCV) are removed from the IGVV at unique patient sequence acceptor sites. These sequences are replaced with the corresponding PDS generated by RT/PCR from patient viral RNA obtained from plasma or serum or cells. In yet another embodiment, in the case of IRES inhibitors, the sequences spanning the IRES (nucleotides 1-709 of the H strain of HCV) are removed from the IGVV at unique patient sequence acceptor sites and are replaced with corresponding PDS generated by RT/PCR from patient viral RNA obtained from plasma or serum or cells.

Drug Susceptibility Tests

Drug susceptibility tests can be carried out with a resistance test vector prepared as described above (either as DNA or RNA) by transfection, using either a one cell assay or a two cell assay. Transfection of host cells with a resistance test vector produces HCV viral particles comprising an encapsidated indicator gene RNA.

Replicate transfections can be performed on a series of packaging host cell cultures maintained either in the presence or absence of an anti-viral agent or in increasing concentrations of the anti-HCV agent (e.g., an HCV NS3/4A protease inhibitor, NS5B polymerase inhibitor, or IRES inhibitor). After maintaining the packaging host cells for an appropriate time (e.g., several hours or several days) in the presence or absence of the anti-HCV agent, the level of drug susceptibility is assessed by measuring indicator gene expression either directly in the host packaging cell lysates or in isolated HCV particles obtained by harvesting the host packaging cell culture media. Alternative approaches can be used to evaluate drug susceptibility in the cell lysates and the isolated HCV particles.

In another embodiment, referred to as the one cell assay (see Section 5.4), drug susceptibility is assessed by measuring luciferase expression or activity in the transfected packaging host cells in the presence or absence of anti-viral agent. A reduction in luciferase activity observed for cells transfected in the presence of a given anti-viral agent, or combination of agents, as compared to a control run in the absence of the anti-viral agent (s), can be used to calculate the inhibiting constant (K₁) of that agent or to generate a sigmoid curve relating the log of the concentration of the anti-viral agent to luciferase activity.

In a second embodiment, referred to as the two cell assay (see Section 5.4), drug susceptibility is assessed by measuring luciferase gene expression and/or activity, in the target host cells following infection with HCV particles obtained by transfecting host cells. At the time of transfection or infection, depending on the drug target, the appropriate concentration of the anti-viral drug is added to the host or target cell cultures. Several days following the infection, the target host cells are lysed and luciferase expression is measured. A reduction in luciferase expression is observed for cells infected in the presence of drugs which inhibit HCV replication, for example by inhibiting either the protease (NS3/4A) or RDRP (NS5B) activities of HCV as compared to a control run in the absence of the drug.

In a third embodiment, in which changes in RNA structure are used as an indicator, drug susceptibility is assessed by measuring the level of HCV RNA replication that has occurred within the transfected host cells. In host cells transfected with the DNA of the HCV viral vector, RNA is transcribed as positive (MRNA) sense RNA, which can then serve as a template for the production of antisense cRNA by the action of NS5B polymerase.

Alternatively, positive sense RNA treated with DNAse to remove residual template DNA is transfected, which is translated and serves as a template for antisense cRNA synthesis. To measure HCV RNA replication, RNA is isolated from the transfected cells and used as template for strand-specific RT-PCR using an RT primer designed to hybridize specifically to antisense HCV RNA. The cDNA reaction is treated with RNAse to prevent reverse transcription of positive sense RNA using the PCR primers, and the cDNA is amplified by PCR. Formation of the amplification target cDNA of positive sense within the transfected cells follows initiation of HCV RNA replication resulting in the formation of antisense RNA. Anti-viral agents that inhibit HCV RNA replication (i.e., those that inhibit RNA-dependent RNA polymerase activity), or production of an active form of the polymerase (by the NS3/4A protease), will limit the formation of the RNA target sequence, which is measured as a decrease in the amplified DNA product using any one of a number of quantitative amplification assays known to one of ordinary skill in the art.

In an alternative embodiment in which changes in RNA structure are used as the indicator, the 5′ exonuclease activity of the amplification enzyme (e.g., Taq polymerase) is measured rather than the production of amplified DNA (Heid et al., 1996, Genome Research 6:986-994). The 5′ exonuclease activity is measured by monitoring the nucleolytic cleavage of a fluorescently tagged oligonucleotide probe capable of binding to the amplified DNA template region flanked by the PCR primer binding sites. The performance of this assay is dependent on the close proximity of the 3′ end of the upstream primer to the 5′ end of the oligonucleotide probe. When the primer is extended it displaces the 5′ end of the oligonucleotide probe such that the 5′ exonuclease activity of the polymerase cleaves the oligonucleotide probe.

Drug Screening

Drug screening can be performed as follows. Replicate transfections can be performed on a series of packaging host cell cultures maintained either in the absence or presence of potential anti-viral compounds (e.g., candidate HCV NS3/4A protease or NS5B polymerase inhibitors). After maintaining the transfected host cells for up to several days in the presence or absence of the candidate anti-viral drugs the level of inhibition of RNA replication can be assessed by measuring indicator gene expression either directly in the transfected host cell lysates, or in isolated HCV particles obtained by harvesting the host transfected cell culture media, or in target cells which are infected with the isolated HCV particles. Either RNA detection or indicator gene activity methods, described above, can be used to evaluate potential anti-HCV drug candidates.

5.8.2 HCV Drug Susceptibility Test Using RTVs Comprising Patient-Derived Segment(s) and a Functional Indicator Gene Expressed From an Internal Ribosomal Initiation Sequence

The present invention provides an assay for determining the susceptibility of HCV to anti-viral agents using RTVs comprising patient-derived segment(s) and a functional indicator gene expressed from an internal ribosomal initiation sequence. The assay optionally includes construction of an IGVV, and/or construction of a RTV.

Construction of Indicator Gene Viral Vector

Initiation of translation of the HCV polyprotein occurs via a cap-independent internal initiation mechanism. The 5′ end of the viral RNA, comprising the untranslated region (UTR) and a portion of the C open reading frame, comprises a sequence and/or structure which directs cap-independent translation initiation (Tsukiyama-Kohara et al, 1992, J Virol. 66:1476,; Wang et al, 1993, J Virol. 67:3338; Lu and Wimmer, 1996, PNAS 93:1412). Other viruses such as poliovirus (PV) (Pelletier and Sonenberg, 1988, Nature, 334, 320-325), encephalomyocarditis virus (EMCV) Wang et al., 1989, J. Virol. 63, 1651-1660), rhinovirus (RV) (Rohll et al., 1994, J. Virol. 68, 4384-4391), hepatitis A virus (HAV) (Brown et al., 1994, J. Virol. 68, 1066-1074; Glass et al., 1993, Virol. 193, 842-852), as well as the pestivirus, bovine viral diarrhea virus (BVDV) (Poole et al., 1995, Virology, 189, 285-292) to which HCV is closely related, employ similar mechanisms for translation initiation, although the sequences which serve as the internal ribosome entry site (RES) are different for each virus. Some cellular mRNAs are also known to initiate translation internally via an IRES (Macejak and Sarnow, 1991, Nature, 353, 90-94). These RNA elements have been shown to be capable of directing translation initiation when located in between two open reading frames, as well as at the 5′ end of RNAs. These bi-cistronic RNAs can be used to obtain expression of two proteins from the same RNA by independently directing the translation of both open reading frames.

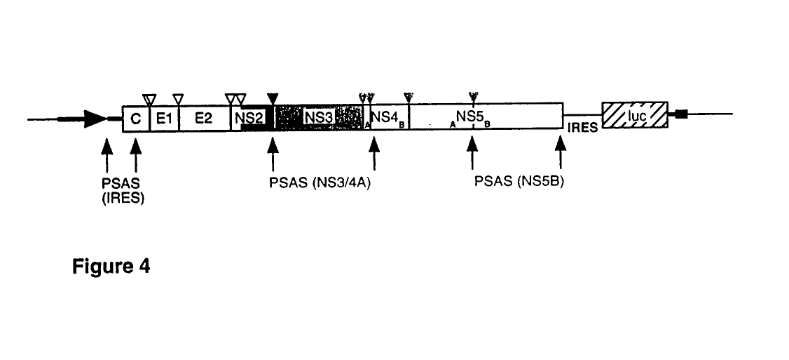

Indicator gene viral vectors comprising a functional IG expressed from an internal ribosomal initiation sequence can be constructed by inserting the open reading frame for an indicator gene, for example, luciferase, in a cDNA construct, comprising all or part of the HCV genome, as a second cistronic element preceded by an IRES. Insertion of the IRES (either the native HCV 5′ UTR or that of another virus) and luciferase downstream of the HCV polyprotein provides for luciferase gene expression independently of that of HCV proteins (see FIG. 4). FIG. 4 is a diagrammatic representation of the resistance test vector (pXHCV-IRESluc) comprising an IRES element for luciferase translation. The IRES can be the native HCV IRES, or derived from other viruses which use such elements for internal initiation of their mRNAs. Expression of luciferase occurs by internal initiation of translation from the bicistronic RNA in the cytoplasm of transfected cells. The IGVV thus can comprise the following elements in a 5′ to 3′ orientation: a promoter sequence, the HCV 5′ UTR, the complete HCV polyprotein coding sequence, an IRES, the luciferase coding region, the HCV 3′ UTR, and a transcription terminator.

In one embodiment, the IGVV comprises a eukaryotic promoter at the 5′ end of the HCV sequences for the production of RNA in transfected cells, and a transcription terminator at the 3′ end. Examples of transcription promoters include, but are not limited to, the CMV intermediate-early promoter, or the SV40 promoter; examples of transcription terminators include, but are not limited to, the transcription terminator/polyadenylation signals found in SV40 or the human β-globin gene (see FIG. 3B).

In a second embodiment, the promoter is a promoter for bacteriophage RNA polymerases such as T7, T3, or SP6, and the terminator is a sequence signaling termination of transcription that is recognized by the polymerase, or a self-cleaving ribozyme (e.g., see Perotta et al., 1991, Nature 350:434-36; Chowrira et al., 1994, J. Biol. Chem. 269: 25864; Wadkins et al., 2002, Cell Mol. Life Sci. 59:112-25). The IGVV is transfected as DNA into cells expressing the RNA polymerase in the cytoplasm. Such expression can be achieved by several methods including, but not limited to, cotransfection with a polymerase expression vector, infection with a recombinant vaccinia virus expressing the polymerase (Fuerst et al., 1986, PNAS 83:8122), and by previously establishing a cell line permanently expressing the polymerase (see FIG. 3C). The IGVV of this embodiment additionally can comprise a functional 3′ end sequence, so that the transcribed RNA can comprise a functional 3′ end sequence.

In a third embodiment, the IGVV with a bacteriophage RNA polymerase promoter at the 5′ end and a terminator sequence at the 3′ end is transcribed in vitro and the nucleic acid representing the IGVV is transfected as RNA. The terminator can be a specific sequence recognized by the bacteriophage RNA polymerase as a termination site or a self-cleaving ribozyme (Perotta et al., 1991, Nature 350:434-36; Chowrira et al., 1994, J. Biol. Chem. 269: 25864; Wadkins et al., 2002, Cell Mol. Life Sci. 59:112-25), or, the terminator can be a restriction endonuclease site allowing for linearization of the DNA template prior to transcription (see FIG. 3D). In this embodiment the vector also comprises a functional 3′ end sequence.

Construction of Resistance Test Vectors

Resistance test vectors comprising a functional indicator gene expressed from an internal ribosomal initiation sequence are constructed from IGVVs described above and patient-derived HCV sequences as described in Section 5.8.1 above. In one embodiment, the IGVV is modified to include a PSAS for the insertion of NS3/4A, NS5B, or IRES comprising PDS (described in Section 5.8.1, see FIG. 3A).

Drug Susceptibility Tests and Drug Screening

Drug susceptibility tests are carried out with a resistance test vector prepared as described above (either as DNA or RNA) by transfection, using either a one cell or two cell assay. Transfection of host cells with a resistance test vector produces HCV viral particles comprising an encapsidated indicator gene RNA. Drug susceptibility tests are performed as described in Section 5.8.1.

Drug screening using an IGVV comprising a functional indicator gene expressed from an internal ribosomal initiation sequence can be performed essentially as described above.

5.8.3 HCV Drug Susceptibility Test Using RTVs Comprising Patient-Derived Segment(s) and a Functional Indicator Gene Expressed From a Replication Defective Minigenome

The present invention provides an assay for determining the susceptibility of HCV to anti-viral agents using RTVs comprising patient-derived segment(s) and a functional indicator gene expressed from a replication defective minigenome. The assay optionally includes construction of an IGVV, and/or construction of a RTV.

Construction of Indicator Gene Viral Vector

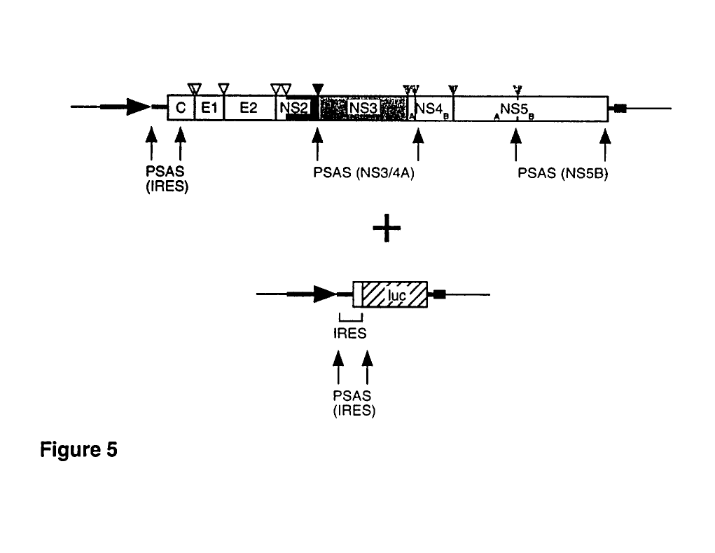

HCV replication-dependent expression of an indicator gene can be achieved by constructing an artificial HCV subgenomic viral vector, or “minigenome,” comprising the HCV 5′ UTR and optionally, an amino-terminal portion of the C open reading frame (required as part of the IRES), an IG, for example luciferase, and the HCV 3′ UTR (see FIG. 5). FIG. 5 is a diagrammatic representation of the resistance test vectors pXHCV and pXIRESluc comprising a positive sense luciferase RNA minigenome. The two constructs can be co-transfected into cells. HCV non-structural proteins expressed from pXHCV can act on both RNAs to replicate and package them. The replicated RNA can be packaged into progeny virions which can then be used for infection of fresh target cells. The target cells also can be infected with HCV or transfected with pXHCV, and the luciferase minigenome can be expressed.

Luciferase can be produced bearing an N-terminal extension derived from the C open reading frame. Alternatively, the ATG at the beginning of the C open reading frame can be mutated so that translation begins at the ATG of luciferase. The 5′ UTR plus the N-terminus of C and 3′ UTR can comprise cis-acting signals required for translation, replication, and packaging of the RNA. The luciferase minigenome can be co-transfected, either as DNA or RNA (see Section 5.8.1, FIGS. 3B-3D), with a full-length or subgenomic helper HCV genomic construct. Replication and packaging of the minigenome RNA into progeny viruses is dependent on the HCV replication machinery, including the NS3/4A protease and NS5B RDRP, produced from the helper HCV genomic RNA, as well as of the cis-acting regulatory elements of the minigenome.

Indicator gene viral vectors comprising a functional indicator gene expressed from a replication defective minigenome and a helper HCV genomic construct can be constructed as follows. The minigenome can comprise the following elements in a 5′ to 3′ orientation: a promoter sequence, the HCV 5′ UTR, the first 24 or 369 nucleotides of the C open reading frame, the luciferase open reading frame, the HCV 3′ UTR, and a transcription terminator. The helper HCV genomic construct can comprise a promoter, the complete HCV cDNA, and a terminator.

In one embodiment, the IGVV comprises a eukaryotic promoter at the 5′ end of the HCV sequences for the production of RNA in transfected cells, and a transcription terminator at the 3′ end. Examples of transcription promoters include, but are not limited to, the CMV intermediate-early promoter, or the SV40 promoter; examples of transcription terminators include, but are not limited to, the transcription terminator/polyadenylation signals found in SV40 or the human β-globin gene (see FIG. 3B).

In a second embodiment, the promoter is a promoter for bacteriophage RNA polymerases such as T7, T3, or SP6, and the terminator is a sequence signaling termination of transcription that is recognized by the polymerase, or a self-cleaving ribozyme (Perotta et al., 1991, Nature 350:434-36; Chowrira et al., 1994, J. Biol. Chem. 269: 25864; Wadkins et al., 2002, Cell Mol. Life Sci. 59:112-25). The IGVV is transfected as DNA into cells expressing the RNA polymerase in the cytoplasm. Such expression can be achieved by several methods including, but not limited to, cotransfection with a polymerase expression vector, infection with a recombinant vaccinia virus expressing the polymerase (Fuerst et al., 1986, PNAS 83:8122), and by previously establishing a cell line permanently expressing the polymerase (see FIG. 3C). The IGVV of this embodiment additionally can comprise a functional 3′ end sequence.

In a third embodiment, the IGVV with a bacteriophage RNA polymerase promoter at the 5′ end and a terminator sequence at the 3′ end is transcribed in vitro and the nucleic acid representing the IGVV is transfected as RNA. The terminator can be a specific sequence recognized by the bacteriophage RNA polymerase as a termination site or a self-cleaving ribozyme (Perotta et al., 1991, Nature 350:434-36; Chowrira et al., 1994, J. Biol. Chem. 269: 25864; Wadkins et al., 2002, Cell Mol. Life Sci. 59:112-25), or, the terminator can be a restriction endonuclease site allowing for linearization of the DNA template prior to transcription (see FIG. 3D). In this embodiment the vector also comprises a functional 3′ end sequence.

Construction of Resistance Test Vectors, Drug Susceptibility Tests and Drug Screening

Resistance test vectors comprising a functional indicator gene expressed from a replication defective minigenome and a helper HCV genomic construct are constructed from the helper HCV genomic construct and patient-derived HCV sequences as described in Section 5.8.1. In one embodiment, the helper HCV genomic construct is modified to include a PSAS for the insertion of NS3/4A or NS5B-comprising PDS (described in Section 5.8.1, see FIG. 3A). For an anti-viral agent that targets the function of the IRES, the PSAS is introduced into the minigenome construct as well.

Drug susceptibility tests using an IGVV system that comprises a functional indicator gene expressed from a minigenome and a helper HCV genomic construct can be carried out with resistance test vectors prepared as described above (either as DNA or RNA) by transfection, using either a one cell or two cell assay. Transfection of host cells with the resistance test vectors (the luciferase minigenome plus the helper HCV genomic construct comprising the PDS) produces HCV viral particles comprising an encapsidated luciferase gene RNA and/or an encapsidated HCV genomic RNA. Drug susceptibility tests can then be performed as described in Section 5.8.1.

Drug screening using an IGVV system that comprises a functional indicator gene expressed from a minigenome and a helper HCV genomic construct can be performed essentially as described in Section 5.8.1 above.

5.8.4 HCV Drug Susceptibility Test Using RTVs Comprising Patient-Derived Segment(s) and a Functional Indicator Gene Expressed From Antisense Replication Defective Minigenomes

The present invention provides an assay for determining the susceptibility of HCV to anti-viral agents using RTVs comprising patient-derived segment(s) and a non-functional indicator gene transfected as antisense replication defective minigenomes. The assay optionally includes construction of an IGVV, and/or construction of a RTV.

Construction of Indicator Gene Viral Vector

Indicator gene viral vectors comprising a non-functional indicator gene transfected as antisense replication defective minigenome and a helper HCV genomic construct can be constructed as follows. The minigenome can comprise the following elements in a 5′ to 3′ orientation: a promoter sequence, the HCV 3′ UTR (in antisense orientation), the luciferase open reading frame (antisense), the first 24 or 369 nucleotides of the C open reading frame (antisense), the HCV 5′ UTR (antisense), and a transcription terminator.

The helper HCV genomic construct can comprise a promoter, the complete HCV cDNA (in sense orientation), and a terminator.

The minigenome can be introduced into the cells as antisense RNA, i.e., as a replicative intermediate RNA copy of the minigenome described above (FIG. 6). FIG. 6 is a diagrammatic representation of the two part resistance test vector pXHCV-ASIRESluc comprising an antisense sense RNA mini-genome. The two constructs can be co-transfected into cells. HCV non-structural proteins expressed from pXHCV act on both RNAs, leading to their replication. Expression in the transfected cells is dependent on the activity of NS5B, production of which is dependent in turn on the action of NS3/4A and the cis-acting regulatory elements such as the IRES. Thus the indicator gene is non-functional until acted upon by the viral replication machinery. In one embodiment, the replicated RNA is packaged into progeny virions which then are used for infection of fresh target cells. In a more particularly defined embodiment, the target cells also are infected with HCV or transfected with pXHCV, and the luciferase minigenome (now with positive sense RNA) is expressed.

In one embodiment, the IGVV comprises a eukaryotic promoter at the 5′ end of the HCV sequences for the production of RNA in transfected cells, and a transcription terminator at the 3′ end. Examples of transcription promoters include, but are not limited to, the CMV intermediate-early promoter, or the SV40 promoter; examples of transcription terminators include, but are not limited to, the transcription terminator/polyadenylation signals found in SV40 or the human β-globin gene (see FIG. 3B).

In a second embodiment, the promoter is a promoter for bacteriophage RNA polymerases such as T7, T3, or SP6, and the terminator is a sequence signaling termination of transcription that is recognized by the polymerase, or a self-cleaving ribozyme (Perotta et al., 1991, Nature 350:434-36; Chowrira et al., 1994, J. Biol. Chem. 269: 25864; Wadkins et al., 2002, Cell Mol. Life Sci. 59:112-25). The IGVV is transfected as DNA into cells expressing the RNA polymerase in the cytoplasm. Such expression can be achieved by several methods including, but not limited to, cotransfection with a polymerase expression vector, infection with a recombinant vaccinia virus expressing the polymerase (Fuerst et al., 1986, PNAS 83:8122), and by previously establishing a cell line permanently expressing the polymerase (see FIG. 3C). The IGVV of this embodiment can additionally comprise a functional 3′ end sequence immediately following the HCV 3′ terminus, so that the transcribed RNA can comprise a functional 3′ end sequence.

In a third embodiment, the IGVV with a bacteriophage RNA polymerase promoter at the 5′ end and a terminator sequence at the 3′ end is transcribed in vitro and the nucleic acid representing the IGVV is transfected as RNA. The terminator can be a specific sequence recognized by the bacteriophage RNA polymerase as a termination site or a self-cleaving ribozyme (Perotta et al., 1991, Nature 350:434-36; Chowrira et al., 1994, J. Biol. Chem. 269: 25864; Wadkins et al., 2002, Cell Mol. Life Sci. 59:112-25), or, the terminator can be a restriction endonuclease site allowing for linearization of the DNA template prior to transcription (see FIG. 3D). In this embodiment the vector also comprises a functional 3′ end sequence.

Construction of Resistance Test Vectors, Drug Susceptibility Tests and Drug Screening

Resistance test vectors comprising a non-functional indicator gene expressed from antisense replication defective minigenome and a helper HCV genomic construct can be constructed from the helper HCV genomic construct and patient-derived HCV sequences as described in Section 5.8.1. The helper HCV genomic construct can be modified to include a PSAS for the insertion of NS3/4A or NS5B-comprising PDS (described in Section 5.8.1, see FIG. 3A). For an anti-viral agent that targets the function of the IRES, the PSAS can be introduced into the minigenome construct as well.

Drug susceptibility tests using resistance test vectors comprising a non-functional indicator gene expressed from a minigenome and a helper HCV genomic construct can be carried out with resistance test vectors prepared as described above (either as DNA or RNA) by transfection, using either a one cell or two cell assay. Transfection of host cells with the resistance test vectors (the luciferase minigenome plus the helper HCV genomic construct comprising the PDS) produces HCV viral particles comprising an encapsidated luciferase gene RNA and/or an encapsidated HCV genomic RNA. Drug susceptibility tests are then performed as described in Section 5.8.1.

Drug screening using resistance test vectors comprising a non-functional indicator gene expressed from a minigenome and a helper HCV genomic construct is performed essentially as described in Section 5.8.1 above.

5.8.5 HCV Drug Susceptibility Test Using RTVs Comprising Patient-Derived Segment(s) and a Functional Indicator Gene Expressed as a Replication Defective Genome

The present invention provides an assay for determining the susceptibility of HCV to anti-viral agents using RTVs comprising patient-derived segment(s) and a functional indicator gene expressed as a replication defective genome. The assay optionally includes construction of an IGVV, and/or construction of a RTV.

Construction of Indicator Gene Viral Vector

Indicator gene viral vectors comprising a functional indicator gene expressed from a replication defective genome and a packaging vector construct can be constructed as follows. The IGVV can comprise the following elements in a 5′ to 3′ orientation: a promoter sequence, the HCV 5′ UTR, the first 24 or 369 nucleotides of the C open reading frame, the indicator gene open reading frame, the NS2 through NS5B portion of the HCV genome (nucleotides 2768-9373 of the H strain of HCV), the HCV 3′ UTR, and a transcription terminator. The packaging vector can comprise a promoter, the C, E1 and E2 open reading frames of HCV (nucleotides 342-2578 of the H strain of HCV), and a terminator. When the indicator gene is luciferase or another cytoplasmic protein, certain modifications can be made to ensure proper processing of the IG-NS2 junction by the host signal peptidase in the endoplasmic reticulum.

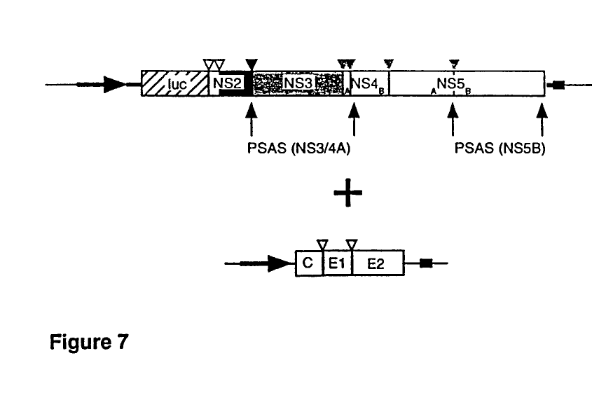

Infectious recombinant virions can be produced from cells transfected with two vectors: an IGVV comprising an IG and the viral non-structural proteins, and a second vector, the packaging vector, comprising the viral structural proteins (C/E1/E2; see FIG. 7). FIG. 7 is a diagrammatic representation of the resistance test vectors pXluc-NSHCV and pXSHCV expressing defective genomic RNAs. The two constructs are cotransfected into cells. Non-structural proteins expressed from pXluc-NSHCV act to replicate the luc-NSHCV RNA and the newly replicated RNA is packaged into virions using structural proteins (C, E1 and E2) from pXSHCV. The progeny virions are then used to infect new cells.

To generate infectious particles, the IGVV DNA (or its corresponding RNA, see Section 5.8.1, FIGS. 3B-3D) can be co-transfected with the packaging vector. Alternatively, particles can be pseudotyped with envelope glycoprotein genes from related flaviviruses such as BVDV or classical swine fever virus (CSFV). The pseudotyped viruses are used, to establish of a cell culture system for single-cycle infection assays. Viruses produced in this manner can then be used to infect target cells, and luciferase expression subsequently measured. This approach has the added advantage of minimizing the amount of manipulations performed with replication competent infectious agents.

In one embodiment, the IGVV comprises a eukaryotic promoter at the 5′ end of the HCV sequences for the production of RNA in transfected cells, and a transcription terminator at the 3′ end. Examples of transcription promoters include, but are not limited to, the CMV intermediate-early promoter, or the SV40 promoter; examples of transcription terminators include, but are not limited to, the transcription terminator/polyadenylation signals found in SV40 or the human P-globin gene (see FIG. 3B).

In a second embodiment, the promoter is a promoter for bacteriophage RNA polymerases such as T7, T3, or SP6, and the terminator is a sequence signaling termination of transcription that is recognized by the polymerase, or a self-cleaving ribozyme (Perotta et al., 1991, Nature 350:434-36; Chowrira et al., 1994, J. Biol. Chem. 269: 25864; Wadkins et al., 2002, Cell Mol. Life Sci. 59:112-25). The IGVV is transfected as DNA into cells expressing the RNA polymerase in the cytoplasm. Such expression can be achieved by several methods including, but not limited to, cotransfection with a polymerase expression vector, infection with a recombinant vaccinia virus expressing the polymerase (Fuerst et al., 1986, PNAS 83:8122), and by previously establishing a cell line permanently expressing the polymerase (see FIG. 3C). The IGVV of this embodiment can additionally comprise a functional 3′ end sequence immediately following the HCV 3′ terminus, so that the transcribed RNA can comprise a functional 3′ end sequence at the 3′ end.

In a third embodiment, the IGVV with a bacteriophage RNA polymerase promoter at the 5′ end and a terminator sequence at the 3′ end is transcribed in vitro and the nucleic acid representing the IGVV is transfected as RNA. The terminator can be a specific sequence recognized by the bacteriophage RNA polymerase as a termination site or a self-cleaving ribozyme (Perotta et al., 1991, Nature 350:434-36; Chowrira et al., 1994, J. Biol. Chem. 269: 25864; Wadkins et al., 2002, Cell Mol. Life Sci. 59:112-25), or, the terminator can be a restriction endonuclease site allowing for linearization of the DNA template prior to transcription (see FIG. 3D). In this embodiment the vector also comprises a functional 3′ end sequence at the 3′ end.

Construction of Resistance Test Vectors, Drug Susceptibility Tests and Drug Screening

Resistance test vectors comprising a functional indicator gene expressed from a replication defective genome and a packaging vector construct are constructed from IGVVs described above and patient-derived HCV sequences as described in Section 5.8.1. The IGVV can be modified to include a PSAS for the insertion of NS3/4A, NS5B, or IRES comprising PDS (described in Section 5.8.1, see FIG. 3A).

Drug susceptibility tests can be carried out with a resistance test vector prepared as described above (either as DNA or RNA) by transfection, using either a one cell or two cell assay. Transfection of host cells with a resistance test vector produces HCV viral particles comprising an encapsidated indicator gene RNA. Drug susceptibility tests can be performed as described in Section 5.8.1.

Drug screening using an IGVV comprising a functional indicator gene expressed from a replication defective genome and a packaging vector construct is performed essentially as described in Section 5.8.1 above.

5.8.6 HCV Drug Susceptibility Test Using RTVs Comprising Patient-Derived Segment(s) and a Functional Indicator Gene in an NS3/4A BVDV Chimeric Viral Vector

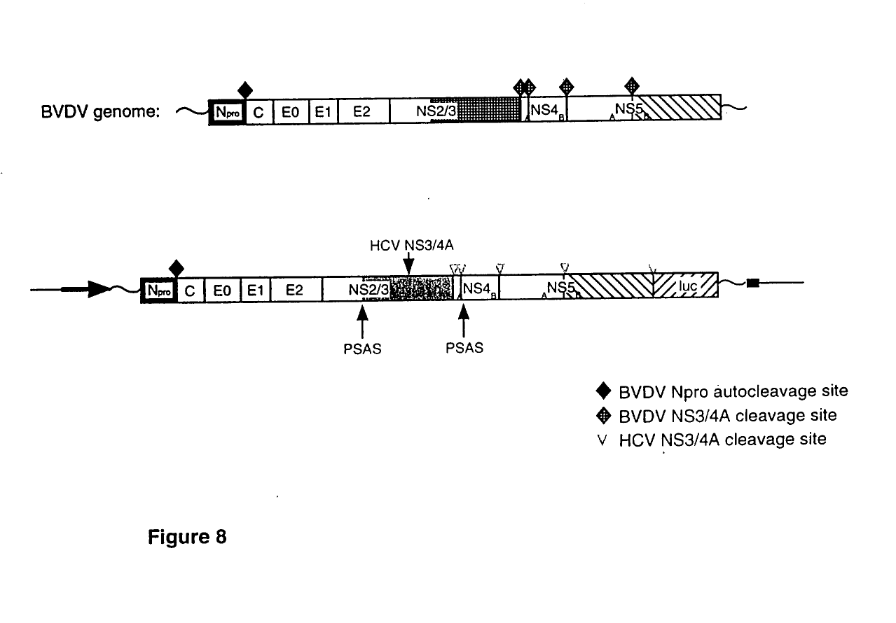

A chimeric IGVV comprising a functional indicator gene and the relevant portion(s) of HCV (for example, the NS3/4A protease domain) can be designed with a backbone of a related virus which replicates well in culture. An example of such a virus is BVDV. A complete cDNA for the genome of BVDV has been assembled and shown to generate infectious RNA by in vitro transcription (Vastsilev et al., 1997, J. Virol. 71:471-478). The BVDV polyprotein is processed in a manner very similar to that of HCV, using both host (signal peptidase) and viral encoded proteases. The chimeric IGVV based on a BVDV backbone can comprise the NS3 protease domain or entire NS3/4A open reading frame of HCV which replaces the corresponding region of BVDV (FIG. 8).

FIG. 8 shows a diagrammatic representation of the genome of BVDV at the top. HCV protease cleavage sites are indicated by triangles, and BVDV protease cleavage sites are represented by crosshatched diamonds (signal peptidase and NS2/3 protease cleavage sites are not shown). FIG. 8 (bottom) shows a diagrammatic representation of the resistance test vector pXBVDV(HCVNS3)luc comprising the BVDV structural protein genes, BVDV NS2, HCV NS3/4A protease, and BVDV NS4B and NS5. The cleavage siles in the nonstructural protein region are altered so that they are recognized by the HCV NS3/4A protease. The luciferase reporter gene is expressed as a fusion with the chimeric polyprotein, and released by cleavage by HCV NS3/4A. Mutating the cleavage sites normally recognized by BVDV NS3 protease to those recognized by HCV NS3/4A, makes the replication of BVDV chimeric RNA and expression of the IG dependent on HCV NS3/4A activity.

Chimeric indicator gene viral vectors comprising a functional indicator gene in an NS3/4A BVDV chimeric viral vector can be constructed as follows. In one embodiment, the IGVV comprises the following elements in a 5′ to 3′ orientation: a promoter sequence, the BVDV 5′ UTR, the C through NS2 regions of BVDV (NADL strain), the NS3/4A region of HCV, the HCV NS4A/4B cleavage site, the BVDV NS4B open reading frame, the HCV NS4B/5A cleavage site, the BVDV NS5A open reading frame, the HCV NS5A/5B cleavage site, the BVDV NS5B open reading frame, the luciferase open reading frame, the BVDV 3′ UTR and a transcription terminator. In another embodiment, the IGVV comprises the luciferase open reading frame preceded by an IRES in a similar configuration to that described in Section 5.8.2. In yet another embodiment, the luciferase gene is expressed from a minigenome similar to that described in Sections 5.8.3 or 5.8.4.

In one embodiment, the IGVV comprises a eukaryotic promoter at the 5′ end of the HCV sequences for the production of RNA in transfected cells, and a transcription terminator at the 3′ end. Examples of transcription promoters include, but are not limited to, the CMV internediate-early promoter, or the SV40 promoter; examples of transcription terminators include, but are not limited to, the transcription terminator/polyadenylation signals found in SV40 or the human β-globin gene (see FIG. 3B).

In a second embodiment, the promoter is a promoter for bacteriophage RNA polymerases such as T7, T3, or SP6, and the terminator is a sequence signaling termination of transcription that is recognized by the polymerase, or a self-cleaving ribozyme (Perotta et al., 1991, Nature 350:434-36; Chowrira et al., 1994, J. Biol. Chem. 269: 25864; Wadkins et al., 2002, Cell Mol. Life Sci. 59:112-25). The IGVV is transfected as DNA into cells expressing the RNA polymerase in the cytoplasm. Such expression can be achieved by several methods including, but not limited to, cotransfection with a polymerase expression vector, infection with a recombinant vaccinia virus expressing the polymerase (Fuerst et al., 1986, PNAS 83:8122), and by previously establishing a cell line permanently expressing the polymerase (see FIG. 3C). The IGVV of this embodiment can additionally comprise a functional 3′ end sequence immediately following the HCV 3′ terminus, so that the transcribed RNA can comprise a functional 3′ end sequence.

In a third embodiment, the IGVV with a bacteriophage RNA polymerase promoter at the 5′ end and a terminator sequence at the 3′ end is transcribed in vitro and the nucleic acid representing the IGVV is transfected as RNA. The terminator can be a specific sequence recognized by the bacteriophage RNA polymerase as a termination site or a self-cleaving ribozyme (Perotta et al., 1991, Nature 350:434-36; Chowrira et al., 1994, J. Biol. Chem. 269: 25864; Wadkins et al., 2002, Cell Mol Life Sci. 59:112-25), or, the terminator can be a restriction endonuclease site allowing for linearization of the DNA template prior to transcription (see FIG. 3D). In this embodiment the vector also comprises a functional 3′ end sequence at the 3′ end.

Construction of Resistance Test Vectors, Drug Susceptibility Tests and Drug Screening

Resistance test vectors comprising a functional indicator gene in an NS3/4A BVDV chimeric viral vector can be constructed from IGVVs described above and patient-derived HCV NS3/4A sequences as described in Section 5.8.1. The IGVV can be modified to include a PSAS for the insertion of NS3/4A-comprising PDS (described in Section 5.8.1, see FIG. 3A).

Drug susceptibility tests can be carried out with a resistance test vector prepared as described above (either as DNA or RNA) by transfection, using either a one cell or two cell assay. Transfection of host cells with a resistance test vector produces HCV viral particles comprising an encapsidated indicator gene RNA. Drug susceptibility tests can be performed as described in Section 5.8.1.

Drug screening using an IGVV comprising a functional indicator gene expressed from an internal ribosomal initiation sequence can be performed essentially as described in Section 5.8.1 above.

5.8.7 HCV Drug Susceptibility Test Using RTVs Comprising Patient-Derived Segment(s) and a Functional Indicator Gene in an NS5B BVDV Chimeric Viral Vector

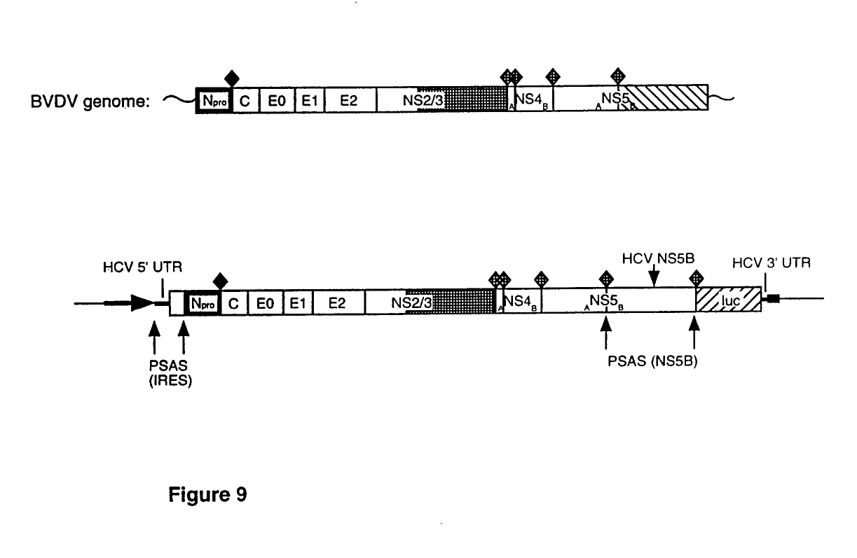

A chimeric IGVV comprising the BVDV structural and non-structural proteins, with the exception of NS5B which is derived from HCV, can be designed with a backbone of BVDV. In addition, the BVDV 5′ and 3′ UTRs are replaced with the corresponding regions from HCV, to ensure recognition by the cognate polymerase (FIG. 9). FIG. 9 is a diagrammatic representation of the resistance test vector pXBVDV(HCVNS5B)luc comprising the BVDV structural protein genes, BVDV NS2, NS3/4A protease, NS4B and NS5A, and HCV NS5B. The cis-acting regulatory elements recognized by the NS5B polymerase, located in the 3′ UTR and 5′ UTR and amino terminal region of the C ORF, are derived from HCV. The luciferase reporter gene is expressed as a fusion with the chimeric polyprotein, and released by cleavage by BVDV NS3/4A.

Indicator gene viral vectors comprising a functional indicator gene in an NS5B BVDV chimeric viral vector can be constructed as follows. In one embodiment, the IGVV comprises the following elements in a 5′ to 3′ orientation: a promoter sequence, the HCV 5′ UTR, sequences from the N-terminus of the HCV C open reading frame required for IRES function, the Npro through NS5A regions of BVDV (NADL strain), the NS5B region of HCV, the luciferase open reading frame, the HCV 3′ UTR, and a transcription terminator. In another embodiment, the IGVV comprises the luciferase open reading frame preceded by an IRES in a similar configuration to that described in Section 5.8.1. In a third embodiment, the luciferase gene is expressed from a minigenome similar to that described in Sections 5.8.3 or 5.8.4.

In one embodiment, the IGVV comprises a eukaryotic promoter at the 5′ end of the HCV sequences for the production of RNA in transfected cells, and a transcription terminator at the 3′ end. Examples of transcription promoters include, but are not limited to, the CMV intermediate-early promoter, or the SV40 promoter; examples of transcription terminators include, but are not limited to, the transcription terminator/polyadenylation signals found in SV40 or the human β-globin gene (see FIG. 3B).

In a second embodiment, the promoter is a promoter for bacteriophage RNA polymerases such as T7, T3, or SP6, and the terminator is a sequence signaling termination of transcription that is recognized by the polymerase, or a self-cleaving ribozyme (Perotta et al, 1991, Nature 350:434-36; Chowrira et al., 1994, J. Biol. Chem. 269: 25864; Wadkins et al., 2002, Cell Mol. Life Sci. 59:112-25). The IGVV is transfected as DNA into cells expressing the RNA polymerase in the cytoplasm. Such expression can be achieved by several methods including, but not limited to, cotransfection with a polymerase expression vector, infection with a recombinant vaccinia virus expressing the polymerase (Fuerst et al., 1986, PNAS 83:8122), and by previously establishing a cell line permanently expressing the polymerase (see FIG. 3C). The IGVV of this embodiment can additionally comprise a functional 3′ end sequence immediately following the HCV 3′ terminus, so that the transcribed RNA can comprise a functional 3′ end sequence at the 3′ end.

In a third embodiment, the IGVV with a bacteriophage RNA polymerase promoter at the 5′ end and a terminator sequence at the 3′ end is transcribed in vitro and the nucleic acid representing the IGVV is transfected as RNA. The terminator can be a specific sequence recognized by the bacteriophage RNA polymerase as a termination site or a self-cleaving ribozyme (Perotta et al., 1991, Nature 350:434-36; Chowrira et al., 1994, J. Biol. Chem. 269: 25864; Wadkins et al., 2002, Cell Mol. Life Sci. 59:112-25), or, the terminator can be a restriction endonuclease site allowing for linearization of the DNA template prior to transcription (see FIG. 3D). In this embodiment the vector also comprises a functional 3′ end sequence at the 3′ end.

Construction of Resistance Test Vectors, Drug Susceptibility Tests and Drug Screening

Resistance test vectors comprising a functional indicator gene in an NS5B BVDV chimeric viral vector can be constructed from IGVVs described above and patient-derived HCV NS5B sequences as described in Section 5.8.1. The IGVV can be modified to include a PSAS for the insertion of NS5B-comprising PDS (described in Section 5.8.1, see FIG. 3A).

Drug susceptibility tests can be carried out with a resistance test vector prepared as described above (either as DNA or RNA) by transfection, using either a one cell or two cell assay. Transfection of host cells with a resistance test vector produces HCV viral particles comprising an encapsidated indicator gene RNA. Drug susceptibility tests can be performed as described in Section 5.8.1.

Drug screening using an IGVV comprising a functional indicator gene expressed from an internal ribosomal initiation sequence can be performed essentially as described in Section 5.8.1 above.

5.8.8 HCV Drug Susceptibility Test Using RTVs Comprising Patient-Derived Segment(s), a Trancriptional Transactivator and a Functional Indicator Gene

An indicator gene viral vector system can be designed involving HCV-dependent expression and release of a transcriptional transactivator which activates the expression of an indicator gene. The indicator gene, for example luciferase, can be introduced as an expression vector into the host cells by transient or stable transfection. The gene encoding the transactivator protein, for example that of HIV-1, tat, can be fused to the HCV polyprotein via a NS3/4A cleavage site linker, in a manner similar to that described for the fusion of luciferase described in Section 5.8.1 (i.e., at the C-terminus or elsewhere). Upon expression of the polyprotein, and dependent on the activity of the NS3/4A protease, tat is cleaved from the polyprotein and activates the transcription of a reporter gene such as luciferase, which is under the control of the HIV-1 long terminal repeat (LTR).

Indicator gene viral vector systems comprising a functional indicator gene comprising patient-derived segment(s), a transcriptional transactivator, and a functional indicator gene can be constructed as follows. The viral vector can comprise the following elements in a 5′ to 3′ orientation: a promoter, the HCV 5′ UTR, the open reading frame for the 3010 amino acid HCV polyprotein, comprising within it the open reading frame for tat, located variously as described in Section 5.8.1, the 3′ UTR, and a transcription terminator. The indicator gene construct can comprise the HIV-1 LTR, the luciferase open reading frame, and a transcription terminator. The indicator gene construct can be co-tranfected with the viral vector, or, preferably, is present as a stable integrated DNA segment in the host cell DNA.

In one embodiment, the IGVV comprises a eukaryotic promoter at the 5′ end of the HCV sequences for the production of RNA in transfected cells, and a transcription terminator at the 3′ end. Examples of transcription promoters include, but are not limited to, the CMV intermediate-early promoter, or the SV40 promoter; examples of transcription terminators include, but are not limited to, the transcription terminator/polyadenylation signals found in SV40 or the human P-globin gene (see FIG. 3B).

In a second embodiment, the promoter is a promoter for bacteriophage RNA polymerases such as T7, T3, or SP6, and the terminator is a sequence signaling termination of transcription that is recognized by the polym erase, or a self-cleaving ribozyme (Perotta et al., 1991, Nature 350:434-36; Chowrira et al., 1994, J Biol. Chem. 269: 25864; Wadkins et al., 2002, Cell Mol. Life Sci. 59:112-25). The IGVV is transfected as DNA into cells expressing the RNA polymerase in the cytoplasm. Such expression can be achieved by several methods including, but not limited to, cotransfection with a polymerase expression vector, infection with a recombinant vaccinia virus expressing the polymerase (Fuerst et al., 1986, PNAS 83:8122), and by previously establishing a cell line permanently expressing the polymerase (see FIG. 3C). The IGVV of this embodiment can additionally comprise a functional 3′ end sequence immediately following the HCV 3′ terminus, so that the transcribed RNA can comprise a functional 3′ end sequence at the 3′ end.

In a third embodiment, the IGVV with a bacteriophage RNA polymerase promoter at the 5′ end and a terminator sequence at the 3′ end is transcribed in vitro and the nucleic acid representing the IGVV is transfected as RNA. The terminator can be a specific sequence recognized by the bacteriophage RNA polymerase as a termination site or a self-cleaving ribozyme (Perotta et al., 1991, Nature 350:434-36; Chowrira et al., 1994, J. Biol. Chem. 269: 25864; Wadkins et al., 2002, Cell Mol. Life Sci. 59:112-25), or, the terminator can be a restriction endonuclease site allowing for linearization of the DNA template prior to transcription (see FIG. 3D). In this embodiment the vector also comprises a functional 3′ end sequence at the 3′ end.

Construction of Resistance Test Vectors, Drug Susceptibility Tests and Drug Screening

Resistance test vectors comprising a functional indicator gene comprising patient-derived segment (s), a transcriptional transactivator, and a functional indicator gene can be constructed from viral vectors described above and patient-derived HCV sequences as described in Section 5.8.1. The viral vector can be modified to include a PSAS for the insertion of PDS comprising the relevant portion of the HCV genome (described in Section 5.8.1, see FIG. 3A).

Drug susceptibility tests can be carried out with a resistance test vector prepared as described above (either as DNA or RNA) by transfection into host cells which comprise the indicator construct and can be performed as described in Section 5.8.1.

Drug screening using indicator gene viral vector systems comprising a functional indicator gene comprising patient-derived segment(s), a transcriptional transactivator, and a functional indicator.gene can be performed. essentially as described in Section 5.8.1.

5.9 Construction of an Indicator Gene Replicon

In one aspect, the present invention provides indicator gene replicons.

Construction of indicator gene replicons used in the invention can include some or all of the following steps, or equivalent steps that would be known to one of skill in the art.

Evaluation of Other Reporter Genes

Indicator gene activity, for example that of green fluorescent protein (“GFP”), can be measured in live cells and is useful for optimizing transfection efficiency (see below). GFP vectors can be tested for the ability to replicate by determining the efficiency and kinetics of indicator gene activity following RNA transfection of Huh-7 cells. Indicator gene replicons of the invention can be constructed with any functional replicon as discussed herein.

Insertion of a Patient Sequence Acceptor Site (“PSAS”)

One or more patient sequence acceptor sites can be introduced at the 5′ and 3′ boundaries of the NS3 and NS5B coding sequences, without changing the predicted protein sequence (e.g., by changing the third positions of codons that do not change the encoded amino acid). Replication efficiency can be monitored to ensure that the modified replicon vectors remain functional and do not lose efficiency.

Modification of the Replicon Vector to Improve Efficiency of Capture of HCV PDS

Following amplification, for example by PCR, of patient sequences (see below), PCR products can be purified, then digested with the restriction enzymes whose recognition sites have been engineered into the PCR primers and which match those introduced into the replicon vector. After further purification, the patient-derived PCR products can be ligated to the digested vector DNA and transformed into competent E. Coli. Any means of ligation and transformation known in the art, including methods taught herein can be used. Following liquid broth culture of sufficient duration, plasmid DNA can be purified from the bacteria using standard methods with modifications as required. This DNA can serve as the template for in vitro transcription.

To improve the efficiency of capture of HCV NS3 and NS5B PDS, the replicon vector can be modified by inserting a bacterial killer gene cassette (e.g., control of cell death B gene (ccdB) or another member of the hok-killer gene family under the control of the E. coli lac operon promoter) in place of NS3 or NS5B (Bernard, 1996, Biotechniques 21:320-3). When placed under the control of the lac operon promoter, transcription of the ccdB killer gene is repressed in bacterial strains, such as JM109, that express the lac I^{Q} repressor. Conversely, bacterial strains that do not express the lac IQ repressor, such as DH5α and Top10, cannot support plasmids that express the ccdB gene product under the control of the lac operon promoter and are killed. DH5a and Top10 cells can be purchased from several vendors, including InVitrogen (Carlsbad, Calif.). Using this selective cloning approach, the parental replicon vector can be propagated in a lac I^{Q}(+) bacterial strain (e.g. Top10 F′, InVitrogen). The vector can be digested with restriction enzymes that remove the ccdB gene cassette and is compatible with the insertion of the HCV PDS. Following ligation of the vector and HCV PDS sequences, a lac I^{Q}(−) strain of bacteria can be transformed. Following transformation, only bacteria with plasmids with HCV PDS inserts will grow. Bacteria with plasmids that retain or reconstitute the ccdB killer gene will not survive. In this way, the population of transformed bacteria can be enriched for plasmids that comprise HCV PDS inserts and lack the parental vector containing the ccdB gene. The structure of the ccdB replicon vector can be confirmed by restriction mapping and DNA sequencing.

Modification of the Replicon Vector to Simplify in vitro Transcription

A self-cleaving ribozyrne, such as that of hepatitis delta virus (Perotta et al., 1991, Nature 350:434-36; Chowrira et al., 1994, J. Biol. Chem. 269: 25864; Wadkins et al., 2002, Cell Mol. Life Sci. 59:112-25), can be introduced into the plasmid at the 3′ end of the HCV sequence. This obviates the requirement for restriction enzyme digestion and purification and facilitates high throughput generation of in vitro transcripts. Short ribozyme sequences can be assembled from oligonucleotides or obtained from other sources. In one embodiment, the ribozme is about 50 to about 125 base pairs. In another embodiment, the ribozme is about 75 to about 100 base pairs. In a preferred embodiment, the ribozme is about 85 base pairs. The transcripts can be analyzed by any means known in the art. In one embodiment, the transcripts are analyzed on an Agilent 2100 BioAnalyzer, an analytical instrument that enables accurate, rapid and convenient assessment yield and quality of RNA or DNA. In another embodiment, the transcripts are analyzed on a denaturing agarose gel stained with ethidium bromide.

5.10 Evaluation of Candidate Cell Lines

Candidate cell lines of the invention can be evaluated for their use in the susceptibility assays of the invention by a process that can include some or all of the following steps, or equivalent steps that would be known to one of skill in the art.

Optimizing the transfection conditions as well as testing the functionality of the indicator gene replicons (neo, luciferase or other any other indicator gene) can be done in a wide variety of human hepatoma cell lines. Examples of hepatoma cell lines that can be used with the invention include HepG2, Hep3B, PLC/PRF/5, Huh6, HLE, SK-Hep1, HepT1, HepT3, and HLF (17, 22, 36). Primary hepatocytes available from several commercial sources, for example, In Vitro Technologies (Baltimore, Md.) and Cambrex (Baltimore, Md.), also can be tested.

Transfection efficiency in the various cell lines can be evaluated in parallel with Huh-7 cells. Various methods of RNA transfection, primarily electroporation and lipofection, can be compared. Electroporation (voltage, capacitance, cell number, etc.) and lipofection (RNA and lipid quantity and relative ratio, type of lipid, and cell density) conditions that give rise to the maximum number of transfected cells can be determined using an HCV IRES-GFP control RNA. Then the functionality of the replicon can be evaluated by selection of replicon-transfected cells comprising a selected marker (e.g., neo) and by luciferase replicon activity at 24-48 hours post-transfection. Efficiency of colony formation and luciferase activity that is approximately equal to that obtained in Huh-7 cells can be considered to be good.

5.11 Patient-Derived Segment (PDS) Amplification Technology

The technology involved in the amplification of a PDS used in the susceptibility assay os the invention can include some or all of the following steps, or equivalent steps that would be known to one of skill in the art.

RNA Purification

Purification of RNA from plasma samples can be performed by one of several methods, including guanidinium isothiocyanate extraction, phenol-chloroform extraction, adsorption to silica-based matrices, or other affinity purification methods known to one of skill in the art. In one embodiment, a solid-phase affinity purification method, including HCV-specific oligonucleotides covalently coupled to magnetic beads is used. Using this approach, all of the RNA captured from an aliquot of plasma (e.g., 1 ml or more) is subjected to reverse transcription (“RT”) by adding reaction components to the magnetic beads. Following the RT reaction, all of the synthesized cDNA is added to the PCR by simply transferring the beads. Other embodiments, including, but not limited to, standard kit-based purification methods, can be used (e.g., Qiagen Qiamp viral RNA kits). RNA yield and quality obtained using various methods, including RT-PCR product yield and specificity on agarose gels and real-time quantitative PCR (“TaqMan”) methods can be evaluated and preferably, the method yielding the optimum yield and quality of RNA is used.

RT and PCR Primer Design

Multiple alignments of nucleotide and amino acid sequences can be used to design candidate primers based on homology in relevant sites (e.g., regions flanking the NS3 and NS5B coding sequences) across a variety of evolutionarily divergent HCV strains.

Alignments are generated using both heuristic and probabilistic models. In the first model, a multiple alignment can be constructed using a process of progressive pairwise comparison. The algorithm parameters can be specified to accommodate different evolutionary models (implemented in Clustal X) (Jeanmougin et al., 1998, Trends Biochem Sci 23:403-5; Thompson et al., 1994, Nucleic Acids Res 22:4673-80). One advantage of this approach is the ability to identify highly conserved sequence blocks across particular phylogenetic groups (i.e., among subtypes). The sequences can also be aligned using an approach based on profile-hidden Markov models (implemented in the program HMMER 2.2) (Eddy, 1998, Bioinformatics 14:755-63). This “Maximum Likelihood” implementation assigns probabilities to various predicted protein structures. based on a simulated annealing model. Preferably, both approaches can be used in order to accommodate for variability both among and within subtypes.

Numerous candidate oligonucleotides can be identified and synthesized for empirical testing. Positions showing variability across subtypes (“degenerate sites”) can be accommodated by equimolar mixing of non-degenerate strain-specific primer sequences post-synthesis.

Candidate RT and PCR primers can be screened empirically against a broad range of reference strains and primary patient isolates to determine their specificity and sensitivity. Panels of HCV-positive plasma with known subtype and viral load are commercially available (e.g., Teragenix, Fort Lauderdale, Fla.). The quantity and specificity of resulting PCR amplification products can be evaluated by any means known to one of skill in the art, including, but not limited to, agarose gel electrophoresis.

RT-PCR Optimization

Each PCR amplicon has unique properties that dictate the performance of various commercially available reverse transcriptase and thermostable polymerase enzymes. RNAseH-minus variant RT such as SuperScript II™ or ThermoScript™ (InVitrogen, Carlsbad, Calif.) provides the best results for use with a solid phase-oligonucleotide based capture methodology for RNA purification, which depends on nascent cDNA hybridized to the RNA (see above). ThermoScripttm has the added advantage of being active at elevated temperatures, which allows for longer transcripts through regions of high secondary structure. ThermoScript™ at 60° C. can, under some conditions, be more efficient than Superscript™ at 45° C. For PCR, many combinations of thermostable polymerase and proofreading enzymes are available which can be assessed for efficiency (amount of DNA produced), sensitivity (lowest input copy number reproducibly amplified), and fidelity (number of mutations introduced per kb of amplified DNA). Enzyme efficiency can be evaluated based on the quantity of PCR product generated and product specificity as visualized, for eaxample, on agarose gels. Sensitivity can be assessed by performing amplification reactions on serial dilutions of a virus sample of know viral load and determining the lowest viral load at which more than 50% of reactions are positive (i.e., by generating a PCR product visible, for example, on ethidium-bromide stained agarose gels).

5.12 Optimization of In Vitro Transcription and RNA Transfection

Some or all of the following steps, or equivalent steps that would be known to one of skill in the art can be used for the optimization of the in vitro transcription and RNA transfection steps that may be performed in order to assess the susceptibility of HCV to an anti-viral agent.

In vitro Transcription

The performance of a phenotypic susceptibility assay depends on the ability to reproducibly generate moderate quantities of high quality RNA from many different templates each comprising a representation of the patient sequences for NS3 or NS5B. This necessitates the development of high throughput methods for preparing plasmid DNA, linearization of plasmid DNA with appropriate restriction enzymes (if a ribozyme approach is not used), and purification of the linear DNA template prior to in vitro transcription. Using a replicon construct comprising a self-cleaving ribozyme simplifies transcription reactions by allowing the use of circular DNA templates, but requires high quality (i.e., RNAse-free) plasmid DNA preparations. Commercially available plasmid preparation kits which are adaptable to high throughput robotic systems can be compared, with and without modifications, by performing transcription reactions from purified DNAs and evaluating the quantity, quality and stability of RNA produced after in vitro transcription. The comparisons or other analyses can be performed by any means known to one of skill in the art. Examples of such comparison means include, but are not limited to, methods using the Agilent 2100 BioAnalyzerm (Agilent Technologies, Palo Alto, Calif.), and denaturing agarose gels stained with ethidium bromide.

RNA Transfection

Transfection of cultured cells with RNA can be performed using any one of several methods, including, but not limited to, DEAE-dextran, lipofection, or electroporation. The best method for each cell line can be determined and optimized empirically. Transfections can be assessed based on overall activity (e.g., by G418-resistant colony formation or initial luciferase activity), kinetics (for example, the ability to discriminate input RNA from replicated RNA), and percentage of cells transfected (e.g., by GFP fluorescence). Better transfection results may be obtained with lipofection and various lipid transfection reagents than electroporation. In addition, the feasibility of increasing the throughput and decreasing the scale of electroporation methods can be evaluated, for example, by using a multi-well electroporation apparatus available from BTX (San Diego, Calif.).

5.13 Distinguishing replicated RNA from Input RNA

The ability to differentiate the reporter activity derived from the input vector from the reporter activity that is dependent on viral vector replication (i.e., viral gene functions) is essential for measuring phenotypic drug susceptibility using vectors that carry a functional indicator/reporter gene. In the HCV system this can be accomplished by exploiting the instability of the replicon RNA in transfected cells. Fortunately, the stability of transfected RNA once inside the cell is low, thus reporter expression detected at later times after transfection (longer than several half lives of the reporter gene and RNA) is likely to be derived principally from intracellular replication of the replicon. The feasibility of this approach has been demonstrated in a time course study performed by Krieger et al. (Krieger et al, 2001, J Virol 75:4614-24) in which the luciferase activity of adapted replicons was compared to a polymerase mutant (“GND”, a point mutation in the NS5B RDRP active site). While transfection of either RNA led to high luciferase activity 4 hours after transfection, the active replicon-transfected cells expressed approximately 100-fold or 1000-fold more luciferase than the GND mutant at 24 or 48 hours post-transfection, respectively (see Krieger et al., 2001, J Virol 75:4614-24). This provides a wide enough dynamic range to detect gradual inhibition of activity by increasing concentrations of an NS3 or NS5B inhibitor. Kinetics of luciferase (or GFP, using a short half life version of this reporter) expression can be determined following transfection of Huh-7 cells by electroporation, lipofection, or other method. Transfections using variable amounts of input RNA can be used to optimize the time at which reporter activity is determined.

5.14 Demonstration of Assay Utility

Generation of Susceptibility Curves to Potential Anti-HCV Drugs

Assays can be performed in the presence of candidate anti-HCV drugs in development. HCV drugs can be obtained commercially from various companies (e.g., Bristol-Myers Squibb, Merck).

The optimal order and precise timing of events (RNA transfection, drug addition and reporter gene detection) can be determined empirically for each inhibitor. Variability in transfection efficiency can be monitored by measuring reporter gene activity shortly (e.g., 4 hours) after transfection in the absence of drug. Activity at a later time point (e.g., 24 or 48 hours) can be determined in the absence of drug or in the presence of drug over a wide range of concentrations. The percent inhibition of the reporter activity at each drug concentration can be plotted against the log10 of drug concentration to generate a sigmoid inhibition curve. Drug toxicity can be measured using standard viability or cytotoxicity assays (e.g., dye exclusion, MTS, ATP).

In order to demonstrate that the assay is capable of measuring changes in susceptibility that correlate with resistance to anti-HCV inhibitors, two approaches can be taken. The first is the construction, by site-directed mutagenesis, of replicons bearing specific amino acid substitutions that reduce drug susceptibility. Such mutations typically can be identified in resistant viruses isolates derived intentionally during in vitro selection experiments or unintentionally in patients during clinical trials. The former can be obtained, for example, by culturing cells stably transfected with the neo replicon in the presence of G418 and low concentrations of the candidate drug.

The second approach involves the assembly of resistance test vectors from patient samples experiencing virologic treatment failure i.e., exhibiting viral load rebound while on therapy following an initial period of suppression. Replicons assembled from viruses from patients failing treatment that have acquired mutations that reduce drug susceptibility generate inhibition curves that are shifted toward higher drug concentrations (to the right with respect to curves generated using the wild-type replicon).

Construction of Multiple RTVs from Patient Samples

Subtype 1b NS3 and NS5B sequences can be amplified using RT-PCR and primers designed to match to the subtype 1b consensus, and inserted into the replicon vector. Activity can be determined as described above i.e., by G418-resistant colony formation efficiency and luciferase activity at 24-48 hours. Alternatively, the replicon can be tailored to a particular patient by identifying regions of general incompatibility by testing smaller portions of each amplicon. This can be accomplished by standard recombinant DNA techniques relying on either naturally occurring or introduced restriction enzyme recognition sites in the patient sequences. A complementary approach can also be used to incorporate additional patient-derived NS gene or regulatory sequences into the replicon backbone. This approach can test the possibility that, for example, a patient derived NS5B interacts well with the 3′ untranslated region derived from its cognate RNA, but not with the replicon vector RNA (Cheng et al., 1999, J. Virol. 73:7044-7049), or that a patient derived NS3 interacts well with the NS4A cofactor from the homologous virus, but not with the NS4A of the replicon (Wright-Minogue et al., 2000, J Hepatol 32:497-504). Alternatively, resistance test vectors can be built from subtypes 1a, 2a, 2b, and 3a.

The methods of the invention take into consideration the possibility that different subtypes of HCV may require different cell lines or modifications of the Huh-7 cell line, or different combinations of known or novel cell culture adaptive mutations in the replicon.

### 6. EXAMPLES

The following examples are provided to illustrate certain aspects of the present invention and do not limit the subject matter thereof in any way.

### 6.1 EXAMPLE 1

### Neomycin Replicons

This example demonstrates that replicons comprising the neomycin resistance-conferring gene are functional.

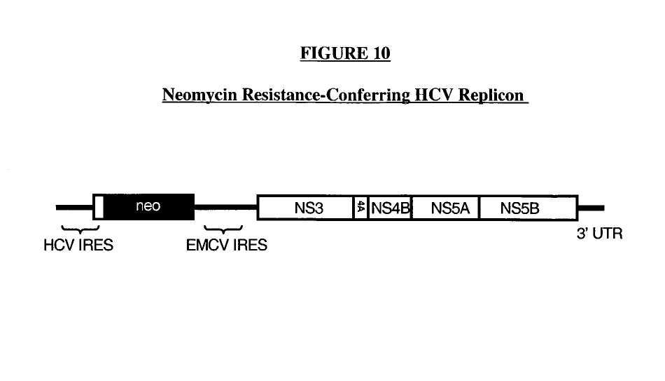

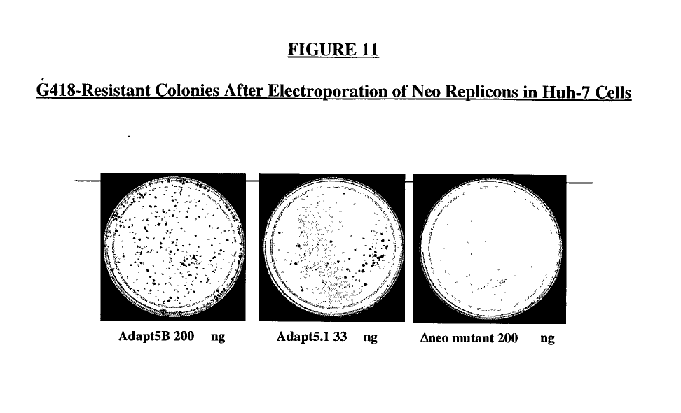

The HCV replicon system of FIG. 10 was used. These replicons had a neomycin resistance marker gene, such as the neomycin phosphotransferase gene (neo) in place of the sequences coding for the structural (C, E1, E2) proteins. Additionally, these replicons had the IRES from encephalomyocarditis virus (EMCV) inserted into the replicon to drive the translation of the HCV NS proteins.

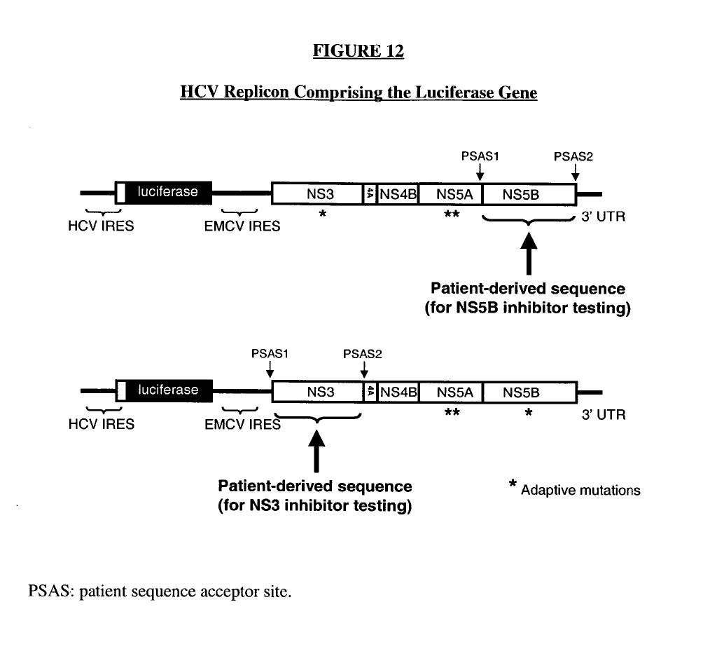

Replication was demonstrated by the generation of cells that grew selectively in the presence of neomycin (G418) (Lohmann et al., 1999, Science 285:110-113). Individual replicons comprised the NS5B R2884G (“Adapt5B”) adaptive mutation (see Lohmann et al., 2001, J Virol 75:1437-1449), or a combination of the NS3 E1202G, T1280I, and NS5A S2179P (“Adapt5.1”) adaptive mutations (see Krieger et al., 2001, J Virol 75:4614-4624), or an NS5B polymerase inactivating mutation (“Δneo”). Various amounts of RNA from the two cell-culture-adapted replicons (Adapt5B and Adapt5.1) or the negative control replicon with an inactive polymerase (Δneo) were electroporated into Huh-7 cells as follows: To generate RNA transcripts from the HCV replicon constructs, 5-10 μg plasmid DNA was digested with Asel and Scal (New England Biolabs, Beverly, Mass.) at 37° C. for 2 hrs. The digested DNA was purified by phenol-chloroform extraction and ethanol precipitation, and resuspended in RNase-free water. T7 transcription reactions were performed using the RIBOMAX™ kit (Promega, Madison Wis.) or the MEGASCRIPT™ kit (Ambion, Austin, Tex.) according to the manufacturers instructions. DNA template was removed by digestion with RNase-free DNase (Promega) at 37° C. for 1 hr. RNA was purified by addition of 60 μl of 2M sodium acetate (pH 4.5), followed by phenol/cholofrom extraction and isopropanol precipitation. Alternatively, RNA transcripts were purified using the MEGACLEAR™ kit (Ambion). RNA transcripts of the HCV replicon were resuspended in RNase-free water, and the concentration and integrity was determined by spectrophotometry and capillary electrophoresis (Bioanalyzer 2100, Agilent Technologies, Palo Alto, Calif.). Subconfluent monolayers of Huh-7 cells were trypsinized and washed twice with phosphate-buffered saline. The concentration of the cells was adjusted to 10⁷ cells/ml in “Cytomix” buffer (120 mM KCl, 0.15 mM CaCl₂, 10 mM K₂HPO₄/KH₂PO₄, 25 mM Hepes, 2 mM EGTA, 5 mM MgCl₂, pH 7.6). RNA was added to 0.4 ml of the cell suspension in a 0.4 cm gap cuvette and the cells were electroporated using a GENE PULSER™ electroporator (Bio-Rad, Hercules, Calif.) at 270V, 975 uF, maximum resistance.Huh-7 cells were electroporated with 1-500 ng HCV replicon RNA plus 17 μg carrier tRNA. Cells were transferred to 10 mls of complete medium, (DMEM containing 10% FBS, 1% Non-essential Amino acids and 1% Penicillin-Streptomycin; Gibco-Invitrogen, Carlsbad, Calif.) in a 10 cm dish. After 24 hrs, the medium was changed to complete media containing either 0.25 mg/ml or 0.50 mg/ml G418 (Gibco-InVitrogen). The medium containing G418 was changed 2-3 times a week for 2-3 weeks. Colonies were stained with crystal violet. (FIG. 11).

### 6.2 EXAMPLE 2

### Luciferase Replicons

This example demonstrates that replicons comprising the luciferase indicator gene are functional.

An HCV replicon system comprising a firefly luciferase reporter gene in addition to the Adapt5B, Adapt5.1 (see FIG. 12 and Example 9, above) adaptive mutations, or the NS5B polymerase inactivating mutation (“GND”) was used. Huh-7 cells were electroporated with 10 μg of RNA plus 17 μg carrier tRNA and transferred into 10 mls of complete medium, and 2 ml of the cells were seeded in 6-well culture plate. At 4, 24, and 48 hours after transfection, cells were washed with PBS and lysed in 350 μl lysis buffer (50 mM Tris phosphate, pH 7.8, 2 mM CDTA, 2 mM DTT, 10% glycerol, 1% Triton X-100). 150 μl of the cell lysate was mixed with 150 μl of luciferase substrate (20 mM TrisHCl, 3.74 mM MgSO₄, 0.1 mM EDTA, 33.3 mM DTT, 0.2 g/l CoenzymeA, 0.4 g/l ATP, 50 mg/l luciferin) in duplicate wells of an opaque 96-well plate, mixed well, and read in a luminometer (Victor2, Perkin Elmer, Boston, Mass.) for 0.5 seconds per well. As shown in FIG. 13, at the 4 hour time point, each transfected cell line produced a high level of luciferase activity. This transient burst of luciferase activity is indepenent of replication. At 24 and 48 hours, significant luciferase activity was detected in cells transfected with either of the adapted replicon RNAs, whereas luciferase activity had returned to background levels in cells transfected with the GND replicon (FIG. 13; all values normalized to the 4 hour activity), indicating that at these later time points luciferase activity is dependent on replication of the replicon. In the graph of FIG. 13, individual points on the graph represent averages (+/−SD) of 3 to 4 independent experiments. The amount of luciferase activity detected at 4 hours post-transfection was about 10-fold lower than reported results (Krieger et al., 2001, J Virol 75:4614-4624).

All references cited herein are expressly incorporated by reference in their entireties.

The examples provided herein, both actual and prophetic, are merely embodiments of the present invention and are not intended to limit the invention in any way.

What is claimed is:

1. A method of determining whether a test hepatitis C virus (HCV) has an altered susceptibility to a compound, comprising
a) contacting a test host cell with the compound, wherein the test host cell comprises a test HCV-derived nucleic acid and an indicator gene, the activity of the indicator gene is affected by the activity of the test HCV-derived nucleic acid such that a change in the activity of the test HCV-derived nucleic acid results in a change in the activity of the indicator gene, and the compound directly or indirectly targets the test HCV-derived nucleic acid or a protein it encodes, and
b) detecting the activity of the indicator gene,
wherein a difference in the activity of the indicator gene in the test host cell contacted with the compound relative to the activity of the indicator gene in a reference host cell contacted with the compound and comprising the indicator gene and a reference HCV-derived nucleic acid, the reference HCV-derived nucleic acid being similar to the test HCV-derived nucleic acid but differing therefrom at one or more nucleotides, indicates that the test HCV has an altered susceptibility to the compound.

2. The method of claim 1 wherein the compound is an anti-viral drug.

3. The method of claim 2 wherein the anti-viral drug is selected from the group consisting of interferon and ribavarin.

4. The method of claim 1 wherein the test HCV-derived nucleic acid and the reference HCV-derived nucleic acid are present in viral vectors comprising one or more of the hepatitis C virus genes C, E1, E2, NS2, NS3, NS4A, NS4B, NS5A or NS5B.

5. The method of claim 1 wherein the test HCV-derived nucleic acid is a patient-derived segment.

6. The method of claim 5 wherein the patient-derived segment comprises one or more of the hepatitis C virus genes C, E1, E2, NS2, NS3, NS4A, NS4B, NS5A or NS5B.

7. The method of claim 1 wherein increased activity of the test host cell relative to the reference host cell indicates the test HCV has decreased susceptibility to the compound.

8. A method of determining whether a compound affects a hepatitis C virus (HCV), comprising
a) contacting a host cell with the compound, wherein the host cell comprises an HCV-derived nucleic acid from the virus and an indicator gene, the activity of the indicator gene is affected by the activity of the HCV-derived nucleic acid such that a change in the activity of the HCV-derived nucleic acid results in a change in the activity of the indicator gene, and the compound directly or indirectly targets the HCV-derived nucleic acid or a protein it encodes, and
b) detecting the activity of the indicator gene,
wherein a difference in the activity of the indicator gene in the host cell contacted with the compound relative to the activity of the indicator gene in the host cell not contacted with the compound indicates that the compound affects the virus.

9. The method of claim 8 wherein the compound is an anti-viral drug.

10. The method of claim 9 wherein the an anti-viral drug is selected from the group consisting of interferon and ribavarin.

11. The method of claim 8 wherein the HCV-derived nucleic acid is present in a viral vector comprising one or more of the hepatitis C virus genes C, E1, E2, NS2, NS3, NS4A, NS4B, NS5A or NS5B.

12. The method of claim 8 wherein the HCV-derived nucleic acid is a patient-derived segment.

13. The method of claim 12 wherein the patient-derived segment comprises one or more of the hepatitis C virus genes C, E1, E2, NS2, NS3, NS4A, NS4B, NS5A or NS5B.

14. The method of claim 8 wherein increased activity of the indicator gene in the test host cell relative to the activity of the indicator gene in the reference host cell indicates the test HCV has decreased susceptibility to the compound.

15. A method of determining whether a patient infected with hepatitis C virus is likely to be susceptible to treatment with an anti-hepatitis C compound comprising
a) contacting a test host cell with the compound, wherein the test host cell comprises a patient-derived viral segment and an indicator gene, the activity of the indicator gene is affected by the activity of the patient-derived viral segment such that a change in the activity of the patient-derived viral segment results in a change in the activity of the indicator gene, and the compound directly or indirectly targets the patient-derived viral segment or a protein it encodes, and
b) detecting the activity of the indicator gene,
wherein an increase in the activity of the indicator gene in the test host cell contacted with the compound relative to the activity of the indicator gene in a reference host cell contacted with the compound and comprising the indicator gene and a reference viral segment, the reference viral segment being similar to the patient-derived viral segment but differing therefrom at one or more nucleotides, indicates that the patient is less likely to be susceptible to treatment with the compound.

16. The method of claim 15 wherein the compound is an anti-viral drug.

17. The method of claim 16 wherein the anti-viral drug is selected from the group consisting of interferon and ribavarin.

18. The method of claim 15 wherein the patient-derived viral segment is present in a viral vector comprising one or more of the hepatitis C virus genes C, E1, E2, NS2, NS3, NS4A, NS4B, NS5A or NS5B.

19. The method of claim 15 wherein the indicator gene is integrated into the host cell genome.

20. The method of Claim 15 wherein the patient-derived viral segment comprises one or more of the hepatitis C virus genes C, E1, E2, NS2, NS3, NS4A, NS4B, NS5A or NS5B.
